(19) United States
(12) Patent Application Publication
(10) Pub. No.: US 2025/0066859 A1
(43) Pub. Date: Feb. 27, 2025

(54) SINGLE-CELL ATAC-SEQ COMPATIBLE WITH 5' RNA-SEQ

(71) Applicant: New York University, New York, NY (US)

(72) Inventors: Sergei KORALOV, New York, NY (US); Ellie IVANOVA, New York, NY (US); Ramin HERATI, New York, NY (US)

(73) Assignee: New York University, New York, NY (US)

(21) Appl. No.: 18/812,510

(22) Filed: Aug. 22, 2024

Related U.S. Application Data
(60) Provisional application No. 63/578,088, filed on Aug. 22, 2023.

(51) Int. Cl. C12Q 1/6886; C12Q 1/6874

(52) U.S. Cl. CPC C12Q 1/6886; C12Q 1/6874; C12Q 2600/156; C12Q 2600/158

(57) ABSTRACT

The invention relates to methods for simultaneously measuring transcription, chromatin accessibility, surface markers, and immune receptor sequence of a biological sample at the single-cell level, comprising one or more cells.

### CROSS REFERENCE TO RELATED APPLICATIONS

This application claims priority to, and the benefit of, U.S. Provisional Application No. 63/578,088 filed on Aug. 22, 2023, the disclosure of which is herein incorporated by reference in its entirety.

### STATEMENT REGARDING FEDERALLY SPONSORED RESEARCH

This invention was made with government support under TR001445 awarded by the National Institutes of Health. The government has certain rights in the invention.

### SEQUENCE LISTING

The instant application contains a Sequence Listing which has been submitted electronically in XML file format and is hereby incorporated by reference in its entirety. Said XML copy, created on Aug. 9, 2024, is named 243735_000383_SL.xml and is 10,047 bytes in size.

### FIELD OF THE INVENTION

The invention relates to methods for simultaneously measuring transcription, the 5′ end of RNA transcripts, chromatin accessibility, surface markers, and immune receptor sequence of a biological sample at the single-cell level, comprising one or more cells.

### BACKGROUND

Cancer development and progression is influenced not only by tumor-intrinsic genetic and epigenetic changes, but also by the dynamic interaction of the malignant cells with the tumor microenvironment (TME). Reciprocal signaling between cancer cells and immune cells in the TME can alter antitumor immune responses and regulate disease progression. While single-cell characterization of TME in a variety of tumors has dramatically advanced the understanding of the evolution of malignant disease and of the anti-tumor immune responses in cancer, this type of analysis often forces investigators to make difficult decisions regarding how much precedence to give to studying immune components of TME and whether to focus on genetic or epigenetic triggers of disease. Because repertoire profiling requires capture of the 5′ end of transcripts, there is currently no platform available that enables profiling of chromatin accessibility alongside B and T lymphocytes antigen repertoire analysis.

### SUMMARY OF THE INVENTION

In one aspect, the invention provides a method for tagmentation of genomic DNA in a cell, the method comprising: a) permeabilizing the cell; and b) incubating the permeabilized cell with a transposome assembly comprising a transposase, a first oligonucleotide, and a second oligonucleotide comprising a 3′ single-stranded overhang on the non-transfer strand, wherein the transposase makes double-strand breaks in genomic DNA and attaches adaptors at the ends of the genomic DNA fragments that are produced, thereby producing tagmented genomic DNA with a transposase recognition site and a complementary sequence to a capture sequence of a third oligonucleotide, the complementary sequence located at the single stranded 3′ end of the non-transfer strand of the tagmented genomic DNA.

In some embodiments, step (a) and step (b) are performed sequentially.

In some embodiments, step (a) and step (b) are performed simultaneously.

In some embodiments, the method further comprises generating the transposome assembly prior to step (b) by a method comprising: i) providing the first oligonucleotide comprising a free 5′ phosphate at a 5′ end of the first oligonucleotide and the transposase recognition site; ii) providing the second oligonucleotide comprising a free 5′ phosphate at a 5′ end of the second oligonucleotide, the transposase recognition site, and a sequence complementary to the capture sequence of the third oligonucleotide at the 3′ end of the second oligonucleotide; iii) providing a fourth oligonucleotide comprising a free 5′ phosphate at a 5′ end of the fourth oligonucleotide, the transposase recognition site, and a unique sequence at the 3′ end of the fourth oligonucleotide; iv) annealing the first oligonucleotide and second oligonucleotide to one another to form a first adaptor that contains a double-stranded transposase recognition site and a single-stranded 3′ overhang on a non-transfer strand, the single-stranded 3′ overhang comprising the sequence complementary to the capture sequence of the third oligonucleotide; v) annealing the first oligonucleotide and fourth oligonucleotide to one another to form a second adaptor that contains a double-stranded transposase recognition site and a single-stranded 3′ overhang on a non-transfer strand, the single-stranded 3′ overhang comprising the unique sequence; and vi) incubating the first adaptor and the second adaptor with the transposase to form the transposome assembly.

In some embodiments, the second oligonucleotide comprises a modification at the 3′ end to prevent degradation of the oligonucleotide, optionally wherein the modification is one or more phosphorothioate bonds.

In some embodiments, the transposase is a Tn5 transposase or a hyperactive Tn5 transposase or a functional mutant or derivative thereof, or a MuA transposase or a functional mutant or derivative thereof, and wherein the transposase recognition site is a Tn5 recognition site or a MuA recognition site comprising a R1 sequence and a R2 sequence.

In some embodiments, the transposase is a Tn5 transposase or a hyperactive Tn5 transposase or a functional mutant or derivative thereof, and wherein the transposase recognition site is the Tn5 recognition site.

In some embodiments, the method further comprises purifying the tagmented genomic DNA fragments.

In some embodiments, the method further comprises gap-filling the tagmented genomic DNA fragments.

In some embodiments, the gap-filling is performed by a non-hot start non-gap filling DNA polymerase.

In some embodiments, the method further comprises ligating the gap-filled tagmented genomic DNA fragments.

In some embodiments, the ligation is performed by a T4 DNA ligase or an E. coli DNA ligase.

In some embodiments, the method further comprises amplifying the ligated gap-filled tagmented genomic fragments DNA with primers.

In some embodiments, the method further comprises preparing an assay for transposase-accessible chromatin (ATAC) library from the amplified genomic DNA fragments.

In another aspect, the invention provides a method for simultaneously measuring the 5′ end of RNA transcripts and chromatin accessibility at the single-cell level in a biological sample comprising one or more cells, the method comprising: a) permeabilizing the cells in the biological sample; b) incubating the permeabilized cells with a plurality of transposome assemblies each comprising a transposase, a first adaptor, and a second adaptor comprising a 3′ single-stranded overhang on the non-transfer strand, wherein the transposase makes double-strand breaks in genomic DNA and attaches the first and second adaptors at the ends of the genomic DNA fragments that are produced, thereby producing tagmented genomic DNA fragments flanked on both sides by a transposase recognition site and a sequence complementary to a capture sequence of a third oligonucleotide, the complementary sequence located at a single stranded 3′ end of the non-transfer strand of tagmented genomic DNA, or flanked on one side by a transposase recognition site and a sequence complementary to a capture sequence of a third oligonucleotide, the complementary sequence located at a single stranded 3′ end of the non-transfer strand of tagmented genomic DNA, and by a transposase recognition site and a unique sequence on the other side; c) incubating the permeabilized cells with a carrier comprising the third oligonucleotide comprising one or more of a barcode sequence, a cell barcode sequence, and a unique molecular identifier (UMI), and d) separating individual cells, each separated cell comprising the carrier comprising the third oligonucleotide, mRNA molecules, and tagmented genomic DNA fragments; e) generating cDNA from the mRNA molecules, the cDNA being captured by the third oligonucleotide attached to the carrier; f) purifying the cDNA and tagmented genomic DNA fragments; g) performing gap-filling on the tagmented genomic DNA fragments; h) ligating to seal nicks in the gap-filled tagmented genomic DNA fragments; i) amplifying the cDNA and the ligated gap-filled tagmented genomic DNA fragments with primers and separating the amplified cDNA from the amplified genomic DNA fragments; j) preparing a cDNA library from the amplified cDNA; and k) preparing an assay for transposase-accessible chromatin (ATAC) library from the amplified genomic DNA fragments.

In some embodiments, the transposase is a Tn5 transposase or a hyperactive Tn5 transposase or a functional mutant or derivative of any said transposase, or a MuA transposase or a functional mutant or derivative thereof, and wherein the transposase recognition site is a Tn5 recognition site or a MuA recognition site comprising a R1 sequence and a R2 sequence.

In some embodiments, the transposase is a Tn5 transposase or a hyperactive Tn5 transposase or a functional mutant or derivative thereof, and wherein the transposase recognition site is the Tn5 recognition site.

In some embodiments, step (a) and step (b) are performed sequentially.

In some embodiments, step (a) and step (b) are performed simultaneously.

In some embodiments, step (g) and step (h) are performed sequentially.

In some embodiments, step (g) and step (h) are performed simultaneously.

In some embodiments, the method comprises generating the transposome assembly comprising the transposase, the first adaptor, and the second adaptor prior to step (b).

In some embodiments, generating the transposome assembly comprises: providing a first oligonucleotide comprising a free 5′ phosphate at a 5′ end of the first oligonucleotide and the transposase recognition site; providing a second oligonucleotide comprising a free 5′ phosphate at a 5′ end of the second oligonucleotide, the transposase recognition site, and the sequence complementary to the capture sequence of the third oligonucleotide at the 3′ end of the second oligonucleotide; providing a fourth oligonucleotide comprising a free 5′ phosphate at a 5′ end of the fourth oligonucleotide, the transposase recognition site, and a unique sequence at the 3′ end of the fourth oligonucleotide; annealing the first oligonucleotide and second oligonucleotide to one another to form the first adaptor that contains a double-stranded transposase recognition site and a single-stranded 3′ overhang on a non-transfer strand, the single-stranded 3′ overhang comprising the sequence complementary to the capture sequence of the third oligonucleotide; annealing the first oligonucleotide and fourth oligonucleotide to one another to form the second adaptor that contains a double-stranded transposase recognition site and a single-stranded 3′ overhang on a non-transfer strand, the single-stranded 3′ overhang comprising the unique sequence; and incubating the first adaptor and the second adaptor with the transposase to form the transposome assembly.

In some embodiments, the second oligonucleotide comprises a modification at the 3′ end to prevent degradation of the oligonucleotide, optionally wherein the modification is one or more phosphorothioate bonds.

In some embodiments, the carrier is a bead or a solid surface.

In some embodiments, the method further comprises prior to step (a), labeling the cells in the biological sample with oligonucleotide-labeled lipids, oligonucleotide-tagged hashing antibodies, or oligonucleotide-tagged antibodies against specific surface markers.

In some embodiments, the cells are labeled with the oligonucleotide-labeled lipids, the oligonucleotide-tagged hashing antibodies, or the oligonucleotide-tagged antibodies against specific surface markers, the method comprising: generating antibody-derived tag (ADT) fragments from the oligonucleotide-tagged antibodies, the ADT fragments being captured by the third oligonucleotide attached to the carrier; purifying the ADT fragments; separating the ADT fragments from the amplified genomic DNA fragments and/or cDNA; amplifying the ADT fragments; and preparing an ADT library from the amplified ADT fragments.

In some embodiments, non-limiting exemplary cell surface markers include CD298, 32 microglobulin, CD45, and MHC class I molecules.

In some embodiments, step (a) further comprises incubation with a RNase inhibitor.

In some embodiments, step (c) further comprises counting the number of cells in the biological sample, resuspending the cells at a concentration of 150-1500 cells/μl, and processing the resuspended cells for bead-based single-cell RNA-sequencing.

In some embodiments, step (b) further comprises inactivating the transposase.

In some embodiments, generating cDNA in step (e) comprises: reverse transcribing the mRNA with a reverse transcriptase enzyme to generate the cDNA fragments; and inactivating the reverse transcriptase enzyme.

In some embodiments, the transposase is inactivated by heating to 65° C., incubating with 0.04% SDS at room temperature, or by the addition of EDTA.

In some embodiments, step (f) comprises forming a plurality of droplets, each droplet comprising an individual cell.

In some embodiments, the gap-filling in step (g) is performed by a non-hot start non-gap filling DNA polymerase.

In some embodiments, the ligation in step (h) is performed by a T4 DNA ligase or an E. coli DNA ligase.

In some embodiments, the cDNA is used to amplify a V(D)J region of transcripts using specific primers to generate a library of V(D)J fragments.

In some embodiments, the cDNA library, ADT library, V(D)J library, and ATAC library are pooled after they are generated.

In some embodiments, the cDNA library, ADT library, V(D)J library, and ATAC library are sequenced in parallel.

In some embodiments, the biological sample is characterized based upon the sequencing readout.

In some embodiments, the biological sample comprises a single cell, and the single readout comprises a single cell readout.

In some embodiments, the method further comprises associating a cDNA fragment, an ADT fragment, a V(D)J fragment, and/or genomic DNA fragment with a single cell in the biological sample based on the cell barcode sequence of third oligonucleotide.

In some embodiments, after a sequencing analysis, the biological sample is assigned to a sample of origin based on a barcode sequence of the carrier and/or of an oligonucleotide-tagged antibody, an oligonucleotide-tagged hashing antibody, or an oligonucleotide-tagged lipid in the ADT library.

In some embodiments, the method further comprises analyzing the sequencing analysis to generate a representation of accessible chromatin, a transcriptomic profile, an immune receptor repertoire, and/or a surface epitope repertoire of a single cell in the biological sample.

In another aspect, the invention provides a method for simultaneously measuring the 5′ end of transcripts, chromatin accessibility, surface markers, and immune receptor sequence of a biological sample comprising one or more cells at the single-cell level, the method comprising: a) labeling cells in the biological sample with oligonucleotide-tagged lipids, oligonucleotide-tagged hashing antibodies, or oligonucleotide-tagged antibodies against specific surface markers; b) permeabilizing the cells in the biological sample; c) incubating permeabilized cells with a plurality of transposome assemblies each comprising a transposase, a first adaptor, and a second adaptor comprising a 3′ single-stranded overhang on the non-transfer strand, wherein the transposase makes double-strand breaks in genomic DNA and attaches the first and second adaptors at the ends of the genomic DNA fragments that are produced, thereby producing tagmented genomic DNA fragments flanked on both sides by a transposase recognition site and a sequence complementary to a capture sequence of a third oligonucleotide, the complementary sequence located at a single stranded 3′ end of the non-transfer strand of tagmented genomic DNA fragments, or flanked on one side by a transposase recognition site and a sequence complementary to a capture sequence of a third oligonucleotide, the complementary sequence located at a single stranded 3′ end of the non-transfer strand of tagmented genomic DNA, and by a transposase recognition site and a unique sequence on the other side; d) incubating the permeabilized cells with a carrier comprising the third oligonucleotide containing one or more of a barcode sequence, a cell barcode sequence, and a unique molecular identifier (UMI); e) separating individual cells, each separated cell comprising the carrier comprising the third oligonucleotide, mRNA molecules, tagmented genomic DNA fragments, and oligonucleotide-tagged antibodies, oligonucleotide-tagged hashing antibodies, or oligonucleotide-tagged lipids from a single cell; f) generating cDNA from the mRNA molecules and generating ADT fragments from the oligonucleotide-tagged antibodies, oligonucleotide-tagged hashing antibodies, or the oligonucleotide-tagged lipids, the cDNA and ADT fragments being captured by the third oligonucleotide attached to the carrier; g) purifying the cDNA, the ADT fragments, and the tagmented genomic DNA fragments; h) performing gap-filling on the tagmented genomic DNA fragments; i) ligating to seal nicks in the gap-filled tagmented genomic DNA fragments; j) amplifying the cDNA, the ADT fragments, and the ligated gap-filled tagmented genomic DNA fragments with primers and separating each of the amplified cDNA, the amplified ADT fragments, and the amplified genomic DNA fragments; k) preparing a cDNA library from the amplified cDNA; 1) preparing an ADT library from the amplified ADT fragments; and m) preparing an ATAC library from the amplified genomic DNA fragments.

In some embodiments, the transposase is a Tn5 transposase or a hyperactive Tn5 transposase or a functional mutant or derivative of any said transposase, or a MuA transposase or a functional mutant or derivative thereof, and wherein the transposase recognition site is a Tn5 recognition site or a MuA recognition site comprising a R1 sequence and a R2 sequence.

In some embodiments, the transposase is a Tn5 transposase or a hyperactive Tn5 transposase or a functional mutant or derivative of any said transposase, and wherein the transposase recognition site is a Tn5 recognition site.

In some embodiments, step (a) and step (b) are performed sequentially.

In some embodiments, step (a) and step (b) are performed simultaneously.

In some embodiments, step (g) and step (h) are performed sequentially.

In some embodiments, step (g) and step (h) are performed simultaneously.

In some embodiments, the method comprises generating the transposome assembly comprising the transposase, the first adaptor, and the second adaptor prior to step (b).

In some embodiments, generating the transposome assembly comprises: providing a first oligonucleotide comprising a free 5′ phosphate at a 5′ end of the first oligonucleotide and the transposase recognition site; providing a second oligonucleotide comprising a free 5′ phosphate at a 5′ end of the second oligonucleotide, the transposase recognition site, and the sequence complementary to the capture sequence of the third oligonucleotide at the 3′ end of the second oligonucleotide; providing a fourth oligonucleotide comprising a free 5′ phosphate at a 5′ end of the fourth oligonucleotide, the transposase recognition site, and a unique sequence at the 3′ end of the fourth oligonucleotide; annealing the first oligonucleotide and second oligonucleotide to one another to form the first adaptor that contains a double-stranded transposase recognition site and a single-stranded 3′ overhang on a non-transfer strand, the single-stranded 3′ overhang comprising the sequence complementary to the capture sequence of the third oligonucleotide; annealing the first oligonucleotide and fourth oligonucleotide to one another to form the second adaptor that contains a double-stranded transposase recognition site and a single-stranded 3′ overhang on a non-transfer strand, the single-stranded 3′ overhang comprising the unique sequence; and incubating the first adaptor and the second adaptor with the transposase to form the transposome assembly.

In some embodiments, the second oligonucleotide comprises a modification at the 3′ end to prevent degradation of the oligonucleotide, optionally wherein the modification is one or more phosphorothioate bonds.

In some embodiments, the carrier is a bead or a solid surface.

In some embodiments, step (a) further comprises incubation with a RNase inhibitor.

In some embodiments, step (c) further comprises counting the number of cells in the biological sample, resuspending the cells at a concentration of 150-1500 cells/μl, and processing the resuspended cells for bead-based single-cell RNA-sequencing.

In some embodiments, step (b) further comprises inactivating the transposase.

In some embodiments, generating cDNA in step (e) comprises: reverse transcribing the mRNA with a reverse transcriptase enzyme to generate the cDNA fragments; and inactivating the reverse transcriptase enzyme.

In some embodiments, the transposase is inactivated by heating to 65° C., incubating with 0.04% SDS at room temperature, or by the addition of EDTA.

In some embodiments, step (f) comprises forming a plurality of droplets, each droplet comprising an individual cell.

In some embodiments, the gap-filling in step (g) is performed by a non-hot start non-gap filling DNA polymerase.

In some embodiments, the ligation in step (h) is performed by a T4 DNA ligase or an E. coli DNA ligase.

In some embodiments, the cDNA is used to amplify a V(D)J region of transcripts using specific primers to generate a library of V(D)J fragments.

In some embodiments, the cDNA library, ADT library, V(D)J library, and ATAC library are pooled after they are generated.

In some embodiments, the cDNA library, ADT library, V(D)J library, and ATAC library are sequenced in parallel.

In some embodiments, the biological sample is characterized based upon the sequencing readout.

In some embodiments, the biological sample comprises a single cell, and the single readout comprises a single cell readout.

In some embodiments, the method further comprises associating a cDNA fragment, an ADT fragment, a V(D)J fragment, and/or genomic DNA fragment with a single cell in the biological sample based on the cell barcode sequence of third oligonucleotide.

In some embodiments, after a sequencing analysis, the biological sample is assigned to a sample of origin based on a barcode sequence of the carrier and/or of an oligonucleotide-tagged antibody, an oligonucleotide-tagged hashing antibody, or an oligonucleotide-tagged lipid in the ADT library.

In some embodiments, the method further comprises analyzing the sequencing analysis to generate a representation of accessible chromatin, a transcriptomic profile, an immune receptor repertoire, and/or a surface epitope repertoire of a single cell in the biological sample.

In an aspect, the invention provides a method for processing a sample, the method comprising: a) contacting a plurality of permeabilized cells labeled with oligonucleotide-tagged antibodies, oligonucleotide-tagged hashing antibodies, or oligonucleotide-tagged lipids with a plurality of transposome assemblies to generate cells comprising a plurality of tagged genomic DNA fragments, wherein the tag is located at a single stranded 3′overhang end of the non-transfer strand of each of the plurality of the tagged genomic DNA fragments; b) generating cDNA from mRNA; c) generating a plurality of antibody-derived tag (ADT) fragments from the oligonucleotide-tagged antibodies, oligonucleotide-tagged hashing antibodies, or the oligonucleotide-tagged lipids; d) partitioning the plurality of permeabilized cells and a carrier comprising a plurality of barcode sequences into a plurality of partitions, wherein at least one of the plurality of partitions comprises (i) one of plurality of permeabilized cells comprising the plurality of tagged genomic DNA fragments, the cDNA, and the plurality of ADT fragments; and (ii) one of the plurality of beads, wherein the carrier comprises a barcode oligonucleotide comprising a first barcode sequence and a second barcode sequence; and e) generating: (i) a first barcoded molecule comprising (1) a sequence of the plurality of tagged genomic DNA fragment, and (2) the barcode oligonucleotide, or a reverse complement thereof; and (ii) a second barcoded molecule comprising (1) a sequence of the cDNA or of a sequence of the plurality of the ADT fragments, and (2) the barcode oligonucleotide, or a reverse complement thereof.

In some embodiments, step (a) comprises: providing a transposase that makes double-strand breaks in genomic DNA and attaches adaptors at the ends of the genomic DNA fragments that are produced; providing a first oligonucleotide comprising a free 5′ phosphate at a 5′ end of the first oligonucleotide and a transposase recognition site; providing a second oligonucleotide comprising a free 5′ phosphate at a 5′ end of the second oligonucleotide, the transposase recognition site, and the sequence complementary to the barcode oligonucleotide at a 3′ overhang end of the second oligonucleotide; annealing the first oligonucleotide and second oligonucleotide to one another to form an adaptor that contains a double-stranded transposase recognition site and a single-stranded 3′ overhang on a non-transfer strand, the single-stranded 3′ overhang comprising the sequence complementary to the barcode oligonucleotide; and incubating the adaptor with the transposase to form the transposome assembly.

In some embodiments, the transposase is a Tn5 transposase or a hyperactive Tn5 transposase or a functional mutant or derivative of any of said transposases, or a MuA transposase or a functional mutant or derivative thereof, and wherein the transposase recognition site is a Tn5 recognition site or a MuA recognition site comprising a R1 sequence and a R2 sequence.

In some embodiments, the transposase is a Tn5 transposase or a hyperactive Tn5 transposase or a functional mutant or derivative of any of said transposases, and wherein the transposase recognition site is a Tn5 recognition site.

In some embodiments, the second oligonucleotide comprises a modification at the 3′ end to prevent degradation of the oligonucleotide, optionally wherein the modification is one or more phosphorothioate bonds.

In some embodiments, step (a) or step (b) further comprises incubation with a RNase inhibitor.

In some embodiments, step (d) further comprises counting the number of cells in the biological sample, resuspending at a concentration of 150-1500 cells/μl, and processing the resuspended biological sample for bead-based single-cell RNA-sequencing.

In some embodiments, step (b) further comprises inactivating the transposase.

In some embodiments, step (b) further comprises: (i) reverse transcribing the mRNA present in the biological sample with a reverse transcriptase enzyme to generate the cDNA fragments and ADT fragments; and (ii) inactivating the reverse transcriptase enzyme.

In some embodiments, the transposase is inactivated by heating to 65° C., incubating with 0.04% SDS at room temperature, or by the addition of EDTA.

In some embodiments, the carrier comprises a bead or a solid surface.

In some embodiments, gap filling using a non-hot start non-strand-displacing DNA polymerase is performed after step (b).

In some embodiments, ligation to seal the nicks in the gap-filled tagmented genomic DNA fragments is performed after the gap filling.

In some embodiments, the ligation is performed by T4 DNA ligase or by E. coli DNA ligase.

In some embodiments, the method further comprises sequencing (i) the first barcoded molecule or a derivative generated from the first barcoded molecule, and (ii) the second barcoded molecule or a derivative generated from the second barcoded molecule to generate a plurality of sequencing reads corresponding to the of genomic DNA fragment, the ADT fragment, and the cDNA.

In some embodiments, the cDNA is used to amplify a V(D)J region of transcripts using specific primers to generate a plurality of V(D)J fragments.

In some embodiments, the cDNA, plurality of ADT fragments, plurality of V(D)J fragments, and plurality of tagged genomic DNA fragments are pooled after they are generated.

In some embodiments, the plurality of cDNA fragments, plurality of ADT fragments, plurality of V(D)J fragments, and plurality of tagged genomic DNA fragments are sequenced in parallel.

In some embodiments, the biological sample is characterized based upon the sequencing readout.

In some embodiments, the biological sample comprises a single cell, and the single readout comprises a single cell readout.

In some embodiments, the method further comprises associating a cDNA fragment, an ADT fragment, a V(D)J fragment, and/or a tagged genomic DNA fragment with a single cell in the biological sample based on the cell barcode sequence of third oligonucleotide.

In some embodiments, the method further comprises associating the tagged fragment of genomic DNA, the cDNA fragment, the ADT fragment, and/or the V(D)J fragment with the permeabilized cell based on the sequencing reads.

In some embodiments, the method further comprises analyzing the sequencing reads to generate a representation of one or more of chromatin accessibility, transcriptome profile, surface markers, and immune cell repertoire of the permeabilized cell.

In some embodiments, one or more of the partitions comprises at most a single cell of the plurality of cells.

In some embodiments, one or more of the partitions comprises at most a single bead of the plurality of beads.

In an aspect, the invention provides a method for generating barcoded nucleic acid fragments, the method comprising: a) permeabilizing a cell; b) incubating the permeabilized cell with a plurality of transposome assemblies each comprising a transposase, a first adaptor, and a second adaptor comprising a 3′ single-stranded overhang on the non-transfer strand, wherein the transposase makes double-strand breaks in genomic DNA and attaches the first and second adaptors at the ends of the genomic DNA fragments that are produced, thereby producing tagmented genomic DNA fragments flanked on both sides by a transposase recognition site and a sequence complementary to a capture sequence of a third oligonucleotide, the complementary sequence located at a single stranded 3′ end of the non-transfer strand of tagmented genomic DNA fragments, or flanked on one side by a transposase recognition site and a sequence complementary to a capture sequence of a third oligonucleotide, the complementary sequence located at a single stranded 3′ end of the non-transfer strand of tagmented genomic DNA, and by a transposase recognition site and a unique sequence on the other side; c) generating cDNA from mRNA; d) purifying the cDNA and tagmented genomic DNA fragments; e) performing gap-filling on the tagmented genomic DNA fragments; f) ligating to seal nicks in the gap-filled tagmented genomic DNA fragments; g) amplifying the cDNA and tagmented genomic DNA fragments with primers and separating the amplified cDNA from the amplified genomic DNA fragments; h) preparing a cDNA library from the amplified cDNA; i) preparing an ATAC library from the amplified genomic DNA fragments; and j) incubating each of the cDNA library and the ATAC library with a carrier comprising the third oligonucleotide to form a complex comprising the carrier and a member of the cDNA library or a member of the ATAC library.

In some embodiments, the transposase is a Tn5 transposase or a hyperactive Tn5 transposase or a functional mutant or derivative of any said transposase, or a MuA transposase or a functional mutant or derivative thereof, and wherein the transposase recognition site is a Tn5 recognition site or a MuA recognition site comprising a R1 sequence and a R2 sequence.

In some embodiments, the transposase is a Tn5 transposase or a hyperactive Tn5 transposase or a functional mutant or derivative thereof, and wherein the transposase recognition site is the Tn5 recognition site.

In some embodiments, step (a) and step (b) are performed sequentially.

In some embodiments, step (a) and step (b) are performed simultaneously.

In some embodiments, step (e) and step (f) are performed sequentially.

In some embodiments, step (e) and step (f) are performed simultaneously.

In some embodiments, the method comprises generating the transposome assembly comprising the transposase, the first adaptor, and the second adaptor prior to step (b).

In some embodiments, generating the transposome assembly comprises: providing a first oligonucleotide comprising a free 5′ phosphate at a 5′ end of the first oligonucleotide and the transposase recognition site; providing a second oligonucleotide comprising a free 5′ phosphate at a 5′ end of the second oligonucleotide, the transposase recognition site, and the sequence complementary to the capture sequence of the third oligonucleotide at the 3′ end of the second oligonucleotide; providing a fourth oligonucleotide comprising a free 5′ phosphate at a 5′ end of the fourth oligonucleotide, the transposase recognition site, and a unique sequence at the 3′ end of the fourth oligonucleotide; annealing the first oligonucleotide and second oligonucleotide to one another to form the first adaptor that contains a double-stranded transposase recognition site and a single-stranded 3′ overhang on a non-transfer strand, the single-stranded 3′ overhang comprising the sequence complementary to the capture sequence of the third oligonucleotide; annealing the first oligonucleotide and fourth oligonucleotide to one another to form the second adaptor that contains a double-stranded transposase recognition site and a single-stranded 3′ overhang on a non-transfer strand, the single-stranded 3′ overhang comprising the unique sequence; and incubating the first adaptor and the second adaptor with the transposase to form the transposome assembly.

In some embodiments, the second oligonucleotide comprises a modification at the 3′ end to prevent degradation of the oligonucleotide, optionally wherein the modification is one or more phosphorothioate bonds.

In some embodiments, the carrier is a bead or a solid surface.

In some embodiments, the method further comprises prior to step (a), labeling the cell with oligonucleotide-labeled lipids, oligonucleotide-tagged hashing antibodies, or oligonucleotide-tagged antibodies against specific surface markers.

In some embodiments, the cell is labeled with the oligonucleotide-labeled lipids, the oligonucleotide-tagged hashing antibodies, or the oligonucleotide-tagged antibodies or against specific surface markers, the method comprising: generating antibody-derived tag (ADT) fragments from the oligonucleotide-tagged antibodies or the oligonucleotide-tagged hashing antibodies, the ADT fragments being captured by the third oligonucleotide attached to the carrier; purifying the ADT fragments; separating the ADT fragments from the amplified genomic DNA fragments and/or cDNA; amplifying the ADT fragments; and preparing an ADT library from the amplified ADT fragments.

In some embodiments, non-limiting exemplary cell surface markers include CD298, 32 microglobulin, CD45, and MHC class I molecules.

In some embodiments, step (a) further comprises incubation with a RNase inhibitor.

In some embodiments, step (b) further comprises inactivating the transposase.

In some embodiments, generating cDNA in step (c) comprises: reverse transcribing the mRNA with a reverse transcriptase enzyme to generate the cDNA fragments; and inactivating the reverse transcriptase enzyme.

In some embodiments, the transposase is inactivated by heating to 65° C., incubating with 0.04% SDS at room temperature, or by the addition of EDTA.

In some embodiments, the gap-filling in step (e) is performed by a non-hot start non-gap filling DNA polymerase.

In some embodiments, the ligation in step (f) is performed by a T4 DNA ligase or an E. coli DNA ligase.

In some embodiments, the cDNA is used to amplify a V(D)J region of transcripts using specific primers to generate a library of V(D)J fragments.

In some embodiments, the cDNA library, ADT library, V(D)J library, and ATAC library are pooled after they are generated.

In some embodiments, the cDNA library, ADT library, V(D)J library, and ATAC library are sequenced in parallel.

In some embodiments, the biological sample is characterized based upon the sequencing readout.

In some embodiments, after a sequencing analysis, the biological sample is assigned to a sample of origin based on a barcode sequence of the carrier and/or of an oligonucleotide-tagged antibody, an oligonucleotide-tagged hashing antibodies, or an oligonucleotide-tagged lipid in the ADT library.

In some embodiments, the method further comprises analyzing the sequencing analysis to generate a representation of accessible chromatin, a transcriptomic profile, an immune receptor repertoire, and/or a surface epitope repertoire of the single cell.

In an aspect, the invention provides a method for processing a sample, the method comprising: a) contacting a plurality of permeabilized cells with a transposome assembly comprising a transposase, a first oligonucleotide, and a second oligonucleotide comprising a 3′ single-stranded overhang on the non-transfer strand, thereby producing tagmented genomic DNA fragments with a transposase recognition site and a complementary sequence to a capture sequence of a third oligonucleotide, the complementary sequence located at the single stranded 3′ end of the non-transfer strand of the tagmented genomic DNA fragments; b) generating cDNA from mRNA; and c) optionally generating: (i) a first barcoded molecule comprising (1) a sequence of the tagged fragment of genomic DNA, and (2) a first barcode, or a reverse complement thereof, the first barcode or reverse complement attached to a bead; and (ii) a second barcoded molecule comprising (1) a sequence of the cDNA fragment, and (2) a second barcode, or a reverse complement thereof, the second barcode or reverse complement attached to the bead.

In some embodiments, step (a) comprises: providing a transposase that makes double-strand breaks in genomic DNA and attaches adaptors at the ends of the genomic DNA fragments that are produced; providing a first oligonucleotide comprising a free 5′ phosphate at a 5′ end of the first oligonucleotide and a transposase recognition site; providing a second oligonucleotide comprising a free 5′ phosphate at a 5′ end of the second oligonucleotide, the transposase recognition site, and a sequence complementary to the first barcode oligonucleotide at a 3′ end of the second oligonucleotide; annealing the first oligonucleotide and second oligonucleotide to one another to form an adaptor that contains a double-stranded transposase recognition site and a single-stranded 3′ overhang on a non-transfer strand, the single-stranded 3′ overhang comprising the sequence complementary to the first barcode oligonucleotide; and incubating the plurality of adaptors with a transposase to form the transposome assembly.

In some embodiments, the second oligonucleotide comprises a modification at the 3′ end to prevent degradation of the oligonucleotide, optionally wherein the modification is one or more phosphorothioate bonds.

In some embodiments, the transposase is a Tn5 transposase or a hyperactive Tn5 transposase or a functional mutant or derivative of any of said transposases, or a MuA transposase or a functional mutant or derivative thereof, and wherein the transposase recognition site is a Tn5 recognition site or a MuA recognition site comprising a R1 sequence and a R2 sequence.

In some embodiments, the transposase is a Tn5 transposase or a hyperactive Tn5 transposase or a functional mutant or derivative of any of said transposases, and wherein the transposase recognition site is a Tn5 recognition site.

In some embodiments, step (b) further comprises incubation with a RNase inhibitor.

In some embodiments, step (a) further comprises counting the cells in the biological sample, resuspending at a concentration of 150-1500 cells/μl, and processing the resuspended biological sample for bead-based single-cell RNA-sequencing before generating the cDNA.

In some embodiments, step (b) further comprises inactivating the transposase.

In some embodiments, step (b) further comprises: reverse transcribing the mRNA present in the biological sample with a reverse transcriptase enzyme to generate the cDNA; and inactivating the reverse transcriptase enzyme.

In some embodiments, the transposase is inactivated by heating to 65° C., incubating with 0.04% SDS at room temperature, or by the addition of EDTA.

In some embodiments, gap filling using a non-hot start non-strand-displacing DNA polymerase is performed after step (b).

In some embodiments, ligation to seal the nicks in the cDNA fragments and tagmented genomic DNA using a ligase.

In some embodiments, the ligase is T4 DNA ligase or E. coli DNA ligase.

In some embodiments, the method further comprises sequencing (i) the first barcoded molecule or a derivative generated from the first barcoded molecule, and (ii) the second barcoded molecule or a derivative generated from the second barcoded molecule to generate a plurality of sequencing reads corresponding to the tagged fragment of genomic DNA and the cDNA.

In some embodiments, the method further comprises associating the tagged fragment of genomic DNA and the cDNA fragment with the permeabilized cell based on the sequencing reads.

In some embodiments, the method further comprises analyzing the sequencing reads to generate a representation of one or more of accessible chromatin, transcriptome profile, and immune cell repertoire of the permeabilized cell.

In some embodiments, one or more of the partitions comprises at most a single cell of the plurality of cells.

In some embodiments, one or more of the partitions comprises at most a single bead of the plurality of beads.

In one aspect, the invention provides a system for processing multiple cells that enables analysis of DNA and mRNA originating from an individual cell in a biological sample, the system comprising: a) a transposome-nucleic acid assembly comprising a transposase and a nucleic acid molecule comprising an adapter sequence located at a single stranded 3′ end on a non-transfer strand of the nucleic acid molecule, wherein said transposome-nucleic acid complex is configured to generate a plurality of adapter-flanked genomic DNA fragments having the adapter sequence located at a single stranded 3′ overhang end of the non-transfer strand of the plurality of adapter-flanked genomic DNA fragments in a plurality of cells, the adapter sequence comprising a sequence complementary to a capture sequence of a third oligonucleotide, the third oligonucleotide comprising one or more of a barcode sequence, a cell barcode sequence, and/or a unique molecular identifier (UMI); b) a reverse transcriptase component configured to generate cDNA from mRNA; c) a gap-filling component configured to fill gaps in the plurality of adapter-flanked genomic DNA fragments; d) a ligation component configured to seal nicks in the plurality of adapter-flanked genomic DNA fragments; and e) a microfluidic device comprising a receiving component that receives the plurality of cells, wherein a cell of the plurality of cells comprises at least one adapter-flanked genomic DNA fragment of the plurality of adapter-flanked genomic DNA fragments, wherein each of the plurality of adapter-flanked genomic DNA fragments is indicative of a region of accessible chromatin within the cell, and a channel structure that partitions (i) the plurality of cells; and (ii) a plurality of barcoded beads into a plurality of droplets, wherein the plurality of barcoded beads comprises a plurality of nucleic acid molecules comprising barcode sequences, and the third oligonucleotide, wherein a droplet of the plurality of droplets comprises: (1) a cell of the plurality of cells comprising at least one adapter-genomic DNA fragment and/or at least one cDNA, and (2) a barcoded bead of the plurality of barcoded beads, wherein the barcoded bead comprises nucleic acid molecules comprising a common barcode sequence, wherein the common barcode sequence of the nucleic acid molecules of the barcoded bead differs from nucleic acid barcode sequences of other barcoded beads in other droplets of the plurality of droplets; and wherein the system is capable of generating a barcoded, adapter-flanked nucleic acid fragment comprising the common barcode sequence, and/or a barcoded cDNA fragment comprising the common barcode sequence.

In some embodiments, the transposome-nucleic acid assembly comprises: the transposase; a first oligonucleotide comprising a free 5′ phosphate at a 5′ end of the first oligonucleotide and a transposase recognition site; and a second oligonucleotide comprising a free 5′ phosphate at a 5′ end of the second oligonucleotide, the transposase recognition site, and a sequence complementary to the capture sequence of the third oligonucleotide at a 3′ end of the second oligonucleotide, wherein the transposome-nucleic acid assembly is further configured to allow annealing of the first oligonucleotide and second oligonucleotide to one another to form a plurality of adaptors that contain a double-stranded transposase recognition site and a single-stranded 3′ overhang on a non-transfer strand, the single-stranded 3′ overhang comprising the sequence complementary to the capture sequence of the third oligonucleotide.

In some embodiments, the second oligonucleotide comprises a modification at the 3′ end to prevent degradation of the oligonucleotide, optionally wherein the modification is one or more phosphorothioate bonds.

In some embodiments, the transposase is a Tn5 transposase or a hyperactive Tn5 transposase or a functional mutant or derivative of any of said transposases, or a MuA transposase or a functional mutant or derivative thereof, and wherein the transposase recognition site is a Tn5 recognition site or a MuA recognition site comprising a R1 sequence and a R2 sequence.

In some embodiments, the transposase is a Tn5 transposase or a hyperactive Tn5 transposase or a functional mutant or derivative of any of said transposases, and wherein the transposase recognition site is a Tn5 recognition site.

In some embodiments, the system further comprises a labeling component configured to label the cell with oligonucleotide-labeled lipids, oligonucleotide-tagged hashing antibodies, or oligonucleotide-tagged antibodies or against specific surface markers. In some embodiments, non-limiting exemplary cell surface markers include CD298, 32 microglobulin, CD45, and MHC class I molecules.

In some embodiments, the cell is labeled with oligonucleotide-tagged antibodies against specific surface markers by a method comprising: generating antibody-derived tag (ADT) fragments from the oligonucleotide-tagged antibodies or the oligonucleotide-tagged hashing antibodies, the ADT fragments being captured by the third oligonucleotide attached to the carrier; purifying the ADT fragments; amplifying the ADT fragments and separating the amplified ADT fragments from the amplified genomic DNA fragments and/or cDNA; and preparing an ADT library from the amplified ADT fragments.

In some embodiments, the system further comprises a RNase inhibitor.

In some embodiments, the transposase is inactivated after generating the plurality of adapter-flanked genomic DNA fragments.

In some embodiments, the reverse transcriptase enzyme is inactivated after generating the plurality of cDNA fragments and ADT fragments.

In some embodiments, the transposase is inactivated by heating to 65° C., incubating with 0.04% SDS at room temperature, or by the addition of EDTA.

In some embodiments, the gap filling polymerase component is a non-hot start non-strand-displacing DNA polymerase.

In some embodiments, the ligation component is T4 DNA ligase or E. coli DNA ligase.

In some embodiments, the nucleic acid molecules comprising the common barcode sequence further comprise a sequence complementary to the adapter sequence and/or the tag.

In some embodiments, the cDNA is used to amplify a V(D)J region of transcripts using specific primers.

In some embodiments, the ligation component is capable of ligating a nucleic acid molecule comprising the common barcode sequence to each of the plurality of adapter-flanked genomic DNA fragments to generate a barcoded, adapter-flanked genomic DNA fragment.

In some embodiments, the system further comprises an amplification component comprising a polymerase enzyme, wherein the amplification component is configured to use a nucleic acid molecule comprising the common barcode sequence and each of the plurality of adapter-flanked genomic DNA fragments, ADT fragments, V(D)J fragments, and cDNA to generate a barcoded, adapter-flanked genomic DNA fragment, a barcoded ADT fragment, a barcoded V(D)J fragment, and/or a barcoded cDNA fragment.

In some embodiments, the adapter sequence comprises a first sequencing primer sequence, wherein the tag comprises a second sequencing primer sequence, and wherein the first sequencing primer sequence is a different sequence than the second sequencing primer sequence.

In some embodiments, the system further comprises a sequencing component comprising a sequencing instrument capable of generating a plurality of sequencing reads corresponding to the adapter-flanked nucleic acid fragment, the ADT fragment, the V(D)J fragment and/or the cDNA fragment.

In some embodiments, the system is further configured to analyze surface protein information, such as surface epitopes and/or immune receptors on the cell.

In some embodiments, the system further comprises a computing component comprising a processor, a user interface, and an electronic display, wherein the computing component is configured to analyze the sequencing reads and surface protein information and display an analysis of nucleic acid sequencing data and surface protein data on the electronic display.

In some embodiments, the analysis comprises a representation of accessible chromatin, the transcriptome profile, the surface epitopes, and/or the immune receptor repertoire of the cell.

In another aspect, the invention provides a system for processing multiple cells that enables analysis of DNA, surface markers, and mRNA originating from an individual cell, comprising: a) a transposome-nucleic acid assembly comprising a transposase and a nucleic acid molecule comprising an adapter sequence located at a single stranded 3′ end on a non-transfer strand of the nucleic acid molecule, wherein said transposome-nucleic acid complex is configured to generate a plurality of adapter-flanked genomic DNA fragments having the adapter sequence located at a single stranded 3′ overhang end of the non-transfer strand of the plurality of adapter-flanked genomic DNA fragments in a plurality of cells, the adapter sequence comprising a sequence complementary to a capture sequence of a third oligonucleotide, the third oligonucleotide comprising one or more of a barcode sequence, a cell barcode sequence, and/or a unique molecular identifier (UMI); b) an oligonucleotide-tagged lipid or oligonucleotide-tagged antibody tagging component configured to label cells with the oligonucleotide-tagged lipids, oligonucleotide-tagged hashing antibodies, or oligonucleotide-tagged antibodies against specific surface markers; c) a reverse transcriptase component configured to generate cDNA from mRNA and to generate antibody derived tag (ADT) fragments from the oligonucleotide-tagged hashing antibodies or the oligonucleotide-tagged antibodies; d) a gap-filling component configured to fill gaps in the plurality of adapter-flanked genomic DNA fragments; e) a ligation component configured to seal nicks in the plurality of adapter-flanked genomic DNA fragments; and f) a microfluidic device comprising a receiving component that receives the plurality of cells, wherein a cell of the plurality of cells comprises at least one adapter-flanked genomic DNA fragment of the plurality of adapter-flanked genomic DNA fragments, wherein each of the plurality of adapter-flanked genomic DNA fragments is indicative of a region of accessible chromatin within the cell, and a channel structure that partitions (i) the plurality of cells; and (ii) a plurality of barcoded beads into a plurality of droplets, wherein the plurality of barcoded beads comprises a plurality of nucleic acid molecules comprising barcode sequences, and the third oligonucleotide, wherein a droplet of the plurality of droplets comprises: (1) a cell of the plurality of cells comprising at least one adapter-flanked genomic DNA fragment, at least one ADT fragment, and/or at least one cDNA, and (2) a barcoded bead of the plurality of barcoded beads, wherein the barcoded bead comprises nucleic acid molecules comprising a common barcode sequence, wherein the common barcode sequence of the nucleic acid molecules and the ADT fragments of the barcoded bead differs from nucleic acid barcode sequences of other barcoded beads in other droplets of the plurality of droplets; and wherein the system is capable of generating a barcoded, adapter-flanked genomic DNA fragment comprising the common barcode sequence, a barcoded ADT fragment comprising the common barcode sequence, and/or a barcoded cDNA comprising the common barcode sequence.

In some embodiments, the transposome-nucleic acid assembly comprises: the transposase; a first oligonucleotide comprising a free 5′ phosphate at a 5′ end of the first oligonucleotide and a transposase recognition site; and a second oligonucleotide comprising a free 5′ phosphate at a 5′ end of the second oligonucleotide, the transposase recognition site, and a sequence complementary to the capture sequence of the third oligonucleotide at a 3′ end of the second oligonucleotide, wherein the transposome-nucleic acid assembly is further configured to allow annealing of the first oligonucleotide and second oligonucleotide to one another to form a plurality of adaptors that contain a double-stranded transposase recognition site and a single-stranded 3′ overhang on a non-transfer strand, the single-stranded 3′ overhang comprising the sequence complementary to the capture sequence of the third oligonucleotide.

In some embodiments, the second oligonucleotide comprises a modification at the 3′ end to prevent degradation of the oligonucleotide, optionally wherein the modification is one or more phosphorothioate bonds.

In some embodiments, the transposase is a Tn5 transposase or a hyperactive Tn5 transposase or a functional mutant or derivative of any of said transposases, or a MuA transposase or a functional mutant or derivative thereof, and wherein the transposase recognition site is a Tn5 recognition site or a MuA recognition site comprising a R1 sequence and a R2 sequence.

In some embodiments, the transposase is a Tn5 transposase or a hyperactive Tn5 transposase or a functional mutant or derivative of any of said transposases, and wherein the transposase recognition site is a Tn5 recognition site.

In some embodiments, the system further comprises a RNase inhibitor.

In some embodiments, the transposase is inactivated after generating the plurality of adapter-flanked genomic DNA fragments.

In some embodiments, the reverse transcriptase enzyme is inactivated after generating the plurality of cDNA fragments and ADT fragments.

In some embodiments, the transposase is inactivated by heating to 65° C., incubating with 0.04% SDS at room temperature, or by the addition of EDTA.

In some embodiments, the gap filling polymerase component is a non-hot start non-strand-displacing DNA polymerase.

In some embodiments, the ligation component is T4 DNA ligase or E. coli DNA ligase.

In some embodiments, the nucleic acid molecules comprising the common barcode sequence further comprise a sequence complementary to the adapter sequence and/or the tag.

In some embodiments, the cDNA fragments are used to amplify a V(D)J region of transcripts using specific primers.

In some embodiments, the ligation component is capable of ligating a nucleic acid molecule comprising the common barcode sequence to each of the plurality of adapter-flanked genomic DNA fragments to generate a barcoded, adapter-flanked genomic DNA fragment.

In some embodiments, the system further comprises an amplification component comprising a polymerase enzyme, wherein the amplification component is configured to use a nucleic acid molecule comprising the common barcode sequence and each of the plurality of adapter-flanked genomic DNA fragments, ADT fragments, V(D)J fragments, and cDNA to generate a barcoded, adapter-flanked genomic DNA fragment, a barcoded ADT fragment, a barcoded V(D)J fragment, and/or a barcoded cDNA.

In some embodiments, the adapter sequence comprises a first sequencing primer sequence, wherein the tag comprises a second sequencing primer sequence, and wherein the first sequencing primer sequence is a different sequence than the second sequencing primer sequence.

In some embodiments, the system further comprises a sequencing component comprising a sequencing instrument capable of generating a plurality of sequencing reads corresponding to the adapter-flanked nucleic acid fragment, the ADT fragment, the V(D)J fragment and/or the cDNA fragment.

In some embodiments, the system is further configured to analyze surface protein information, such as surface epitopes and/or immune receptors on the cell.

In some embodiments, the system further comprises a computing component comprising a processor, a user interface, and an electronic display, wherein the computing component is configured to analyze the sequencing reads and surface protein information and display an analysis of nucleic acid sequencing data and surface protein data on the electronic display.

In some embodiments, the analysis comprises a representation of accessible chromatin, the transcriptome profile, the surface epitopes, and/or the immune receptor repertoire of the cell.

In some embodiments, non-limiting exemplary cell surface markers include CD298, 32 microglobulin, CD45, and MHC class I molecules.

In another aspect, the invention relates to a composition comprising: a transposase; a first oligonucleotide comprising a free 5′ phosphate at a 5′ end of the first oligonucleotide and transposase recognition site; and a second oligonucleotide comprising a free 5′ phosphate at a 5′ end of the second oligonucleotide, the transposase recognition site, and a sequence complementary to the capture sequence of the third oligonucleotide at a 3′ end of the second oligonucleotide on the non-transfer strand, wherein the transposome-nucleic acid assembly is further configured to allow annealing of the first oligonucleotide and second oligonucleotide to one another to form a plurality of adaptors that contain a double-stranded transposase recognition site and a single-stranded 3′ overhang on the non-transfer strand, the single-stranded 3′ overhang comprising the sequence complementary to the capture sequence of the third oligonucleotide, and wherein the composition is configured to produce a plurality of tagmented genomic DNA fragments with a transposase recognition site and a complementary sequence to a capture sequence of a third oligonucleotide, the complementary sequence located at a single stranded 3′ end on a non-transfer strand of the tagmented genomic DNA, wherein the third oligonucleotide is attached to a bead and further comprises at least one of a barcode sequence, a cell barcode sequence, and a unique molecular identifier (UMI).

In some embodiments, the second oligonucleotide comprises a modification at the 3′ end to prevent degradation of the oligonucleotide, optionally wherein the modification is one or more phosphorothioate bonds.

In some embodiments, the transposase is a Tn5 transposase or a hyperactive Tn5 transposase or a functional mutant or derivative of any of said transposases, or a MuA transposase or a functional mutant or derivative thereof, and wherein the transposase recognition site is a Tn5 recognition site or a MuA recognition site comprising a R1 sequence and a R2 sequence.

In some embodiments, the transposase is a Tn5 transposase or a hyperactive Tn5 transposase or a functional mutant or derivative of any of said transposases, and wherein the transposase recognition site is a Tn5 recognition site.

In another aspect, the invention relates to a library of tagmented genomic DNA fragments produced by any of the methods of the disclosure, any of the systems of the disclosure, or any compositions of the disclosure.

In another aspect, the invention relates to the use of any of the methods of the disclosure to analyze a biological sample comprising one or more cells to generate a representation of accessible chromatin, transcriptomic profile, immune receptor repertoire, and surface epitope repertoire of a single cell in the biological sample.

In another aspect, the invention relates to the use of any of the systems of the disclosure to analyze a biological sample comprising one or more cells to generate a representation of accessible chromatin, transcriptomic profile, immune receptor repertoire, and surface epitope repertoire of a single cell in the biological sample.

In another aspect, the invention relates to the use of any of the compositions of the disclosure to generate a representation of accessible chromatin, transcriptomic profile, immune receptor repertoire, and surface epitope repertoire of a single cell in the biological sample.

In another aspect, the invention relates to the use of any of the methods of the disclosure to generate a library of tagmented genomic DNA fragments, a library of ADT fragments, a library of V(D)J fragments, and a library of cDNA fragments.

In another aspect, the invention relates to the use of any of the systems of the disclosure to generate a library of tagmented genomic DNA fragments, a library of ADT fragments, a library of V(D)J fragments, and a library of cDNA fragments.

In another aspect, the invention relates to the use of any of the compositions of the disclosure to generate a library of tagmented genomic DNA fragments.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIGS. 1A-1D show exCITE-seq analysis of blood and tumor microenvironment. Merged tsne plot of aggregated data from blood and tumor sample of a Cutaneous T Cell Lymphoma (CTCL) patient, colored by (FIG. 1A) tissue of origin or (FIG. 1B) cell type. FIG. 1A discloses SEQ ID NOS 5-6, respectively, in order of appearance. Clustering was based on joint visualization of multimodal data. Highlighted cluster of malignant T cells shows a singular TCR clonality in both skin and blood with the same unique CDR3 sequence. FIG. 1C: GEX-based sub-clustering of the malignant clone highlights transcriptional heterogeneity within malignant T cells. FIG. 1D: Pair-wise comparison of differentially expressed genes between skin and blood derived malignant T cells of five CTCL patients. The differentially expressed gene list (adj p-val<0.05, |log₂FC|>=1) was comprised of genes present in at least two out of five CTCL patients analyzed.

FIGS. 2A-2H depict a schematic of standard (3′-compatible) assay for transposase-accessible chromatin with sequencing (ATAC-seq) and 5′-compatible ATAC-seq. FIGS. 2A and 2E: Adaptors. FIGS. 2B and 2F: Tagmentation reaction. FIGS. 2C and 2G: Genomic DNA fragments produced by tagmentation using Tn5 loaded with adaptors with 5′ single-stranded overhang (FIG. 2C) or 3′ single-stranded overhang (FIG. 2G). Consequences of extension of free 3′ end by DNA polymerase of fragments produced in 3′-compatible ATAC-seq (FIG. 2D) and 5′-compatible ATAC-seq (FIG. 2H).

FIGS. 3A-3B show tagmentation fragment size distribution. FIG. 3A: Amplified genomic DNA fragments following Tn5 tagmentation of permeabilized cells on TapeStation. FIG. 3B: Insert size distribution of ATAC library following tagmentation in single cells using Chromium 10×5′ Next GEM v2 kit.

FIGS. 4A-4B show gap-filling of tagmentation fragments. FIG. 4A: Products generated by 3′-compatible scATAC-seq (3′ ATAC-seq). Extension of the free 3′ end flanking the gap using the 5′ adaptor overhang as a template. FIG. 4B: Template generated by 5′-compatible scATAC-seq (5′ ATAC-seq). RT enzyme fills the 9 bp gap and subsequent ligation seals the gap. The crucial adaptor sequences in 3′ and 5′ ATAC-seq are highlighted in blue and red respectively. To avoid displacement of adaptor sequence in 5′ ATAC-seq, RT enzyme without strand displacement activity and lower temperature is used for gap filling.

FIG. 5 shows streptavidin pulldown of biotinylated ATAC fragments. Fragments amplified from supernatant (1) and streptavidin bead-bound fraction (2).

### DETAILED DESCRIPTION

The invention relates to methods for simultaneously measuring one or more of transcription, the 5′ end of RNA transcripts, chromatin accessibility, surface markers, and immune receptor sequence of a biological sample at the single-cell level, comprising one or more cells. The invention also relates to methods for tagmentation of genomic DNA.

Analysis of chromatin accessibility in the course of antigen-specific responses and clonal expansion represents a critical advance for studies related to infection, vaccines, autoimmunity, and cancer. Paired information regarding chromatin changes, transcriptional profile, and V(D)J repertoire of lymphocytes at a single-cell resolution enables visualization of how prior inflammatory responses impacted gene accessibility, how clonally-defined B and T cells are poised to respond to antigen, and how malignant lymphocytes are distinct from non-transformed cells in the TME. Here, a multimodal platform was established for simultaneous profiling of surface antigens, gene expression, immune repertoire, as well as chromatin accessibility in individual cells.

The single-cell ATAC-seq method described herein utilizes a transposase (nonlimiting examples include a Tn5 transposase and functional mutants and derivatives thereof, including a hyperactive Tn5 transposase, collectively referred to herein as a “Tn5 transposase” or “Tn5”, or a MuA transposase or a functional mutant or derivative thereof) to map the distribution of nucleosomes in individual cells. Tn5 binds to synthetic 19 bp mosaic end (ME) recognition sequence and tagments genomic DNA, producing DNA fragments that are tagged with the adapter sequences at both ends. The adapter itself comprises the ME sequence and an additional 3′ single-stranded DNA overhang on the non-transfer strand. In scATAC-seq methods that use bead-based transcript capture, the single-stranded overhang contains a capture sequence that allows it to bind to the bead oligonucleotide and be tagged with a unique molecular identifier (UMI), a barcode, and/or a cell barcode. Currently, Tn5 tagmentation-based applications use adapters with a 5′ single-stranded overhang on the transfer strand of the adapter (i.e., the strand that becomes covalently bound to the genomic DNA). In order to generate fragments compatible with 5′ single-cell methods, the Tn5 enzyme was loaded such that it generated fragments with free 3′ single-stranded overhangs. A method was developed herein for single-cell ATAC-seq that is compatible with 10× Genomics 5′ single-cell RNA sequencing platform.

In a non-limiting example of the methods described herein, live or fixed cells were permeabilized in a buffer containing 20 mM Tris-HCl pH7.4, 150 mM NaCl, 3 mM MgCl2, 0.01% Digitonin, and 1 U/ml RNase inhibitor for 5 minutes on ice and washed in a buffer containing 20 mM Tris-HCl pH7.4, 150 mM NaCl, 3 mM MgCl2. To perform the tagmentation reaction, permeabilized cells were incubated for 60 minutes with Tn5 transposase (Diagenode) loaded with custom adapters containing a mosaic end sequence and a free 3′ single-stranded overhang complementary to the capture sequence of the 10× Genomics bead oligonucleotide. After tagmentation as described above using a hyperactive Tn5 transposase, the cells were counted, and the appropriate number of cells were loaded in a 10× lane according to the manufacturer's protocol and run on a 10× Genomics controller to make the gel bead-in-emulsion (GEM) for barcoding. The reverse transcription reaction was carried out at a lower temperature (37° C.) to avoid strand melting and loss of barcode (e.g., per 10× Genomics protocol). The emulsion was incubated at 53° C. for 45 minutes and at 85° C. for 5 minutes. The cDNA and tagmentation fragments were cleaned up according to the 10× Genomics protocol using 10× Genomics kit reagents or using RLT buffer (Qiagen) and MyOne SILANE beads (Invitrogen). After cleanup, the 9-bp gaps that resulted from tagmentation were filled with a non-hot start non-gap filling DNA polymerase, e.g., T4 DNA polymerase (NEB), by incubating for 15 minutes at 12° C. The reaction was stopped by adding EDTA to a final concentration of 10 mM. Alternatively, the gaps can be filled with KAPA HiFi non-hot start enzyme and buffer at 37 degrees for 10 minutes. The fragments were cleaned up using RLT buffer (Qiagen) and MyOne SILANE beads (Invitrogen) or SPRISelect reagent (Beckman Coulter) and eluted in EB buffer (Qiagen). After gap-filling, the fragments were ligated with a DNA ligase, e.g., with T7 ligase (NEB) in T4 ligase buffer (NEB) for 15 minutes at room temperature, or with E. coli DNA Ligase for 30 minutes at 16° C., followed by inactivation of the E. coli DNA Ligase by incubating at 65° C. for 20 minutes. After ligation, the fragments were cleaned up using SPRISelect reagent and eluted in EB buffer. Alternatively, gap-filling and nick-sealing can be combined in one step by incubating genomic DNA fragments with T4 DNA polymerase and E. Coli ligase at 16° C. for 30 minutes. Fragments were cleaned with SPRISelect reagent and eluted in EB buffer. cDNA and tagmentation fragments can then be amplified, the corresponding libraries prepared for sequencing using 10× reagents, and sequenced using standard Illumina sequencing primers.

Massively parallel single cell methods have advanced from single modal measurements of transcriptomes alone to bimodal platforms capable of simultaneously measuring transcriptome and surface epitopes (CITE-seq, REAP-seq), transcriptome and chromatin accessibility (SNARE-seq, SHARE-seq), and chromatin accessibility and surface epitopes (ASAP-seq, ICICLE-seq) and trimodal platforms that allow simultaneous measurement of transcriptome, surface epitopes, and chromatin accessibility (DOGMA-seq, TEA-seq). The multimodal exCITE-seq platform allows simultaneous measurement of surface epitopes, transcriptional profiling, and immune receptor (V(D)J rearrangement) profiling by utilizing microfluidic droplet-based capture and sequencing of the 5′ end of transcripts instead of the more common poly-A capture and 3′ transcript sequencing. There is currently no available technology that combines all modalities. Simultaneous capture of V(D)J rearrangement and chromatin profiling requires a single-cell ATAC-seq method compatible with 5′-based chemistry which is not currently available. The methods disclosed herein allows unbiased assessment of chromatin accessibility alongside V(D)J repertoire analysis at single cell resolution. Integrated into an exCITE-seq workflow, this 5′-compatible ATAC-seq (5′ATAC-seq) can allow simultaneous measurement of transcriptome, surface epitopes, chromatin accessibility, and profiling of V(D)J rearrangement in single cells. The method described herein can be termed Single-Cell Immune Repertoire, Antigen, Transcription, Chromatin accessibility by sequencing (SCRATCH-seq).

Not all tumor-infiltrating lymphocytes are specific for tumor antigens and often include bystander tissue resident cells. The unique antigen receptors on lymphocytes determine antigen specificity of tumor-infiltrating B and T cells. The present 5′ pipeline enables simultaneous evaluation of antigen receptor rearrangements, transcriptional profiling, and chromatin accessibility. Furthermore, information about TCR/BCR rearrangements can be paired with tetramer straining (using oligonucleotide-tagged B and T antigen tetramers) thereby providing information about receptor identity and specificity at the same time. TCR and BCR repertoire profiling of individual cells can be used to measure lymphocyte dynamics in cancer, as well as after interventions such as cancer immunotherapy to evaluate clinical response to treatment and disease prognosis.

In hematological malignancies (lymphoma/leukemia), TCR and BCR rearrangements are critical for identifying malignant cells against the polyclonal background of healthy polyclonal lymphocytes. Furthermore, BCR sequencing enables the evaluation of Ig somatic hypermutation, which can be a biologically relevant and clinically useful prognostic factor in B cell lymphomas as somatic hypermutations are a hallmark of B lymphocytes that have gone through a germinal-center response.

The ability to detect B cell and T cell clonal responses is also critical for studies of immune responses to infections and vaccines as expansion of clonal cells indicates a productive immune response. For autoimmune diseases, expansion of clonal cells often suggests recognition of self-antigens by autoreactive lymphocytes. Epigenetic changes frequently occur before lymphocyte differentiation into functional T and B cell subsets. On the other hand, the presence of epigenetic scarring can allow researchers to infer the specific stimuli and differentiation signals that individual lymphocytes have previously encountered. In the context of both exogenous challenge/immunogen and self-antigens, the ability to pair TCR/BCR sequencing with transcriptional and epigenetic analysis of individual cells enables critical insight into the molecular changes that occur in antigen-specific lymphocytes.

Epigenetic mechanisms are essential for the normal development and maintenance of cell-specific gene expression patterns. Disruption of these mechanisms can lead to altered gene function and malignant transformation. Epigenetic changes are found in all human cancers, and mutations are frequently observed in genes that modify the epigenome. Global changes in the epigenetic landscape play a significant role in cancer development and progression. Epigenetic therapies are emerging as a viable therapeutic route in hematological malignancies and have shown some promise in solid tumors.

The crosstalk between immune and malignant cells is regulated by multiple factors, including genetic and epigenetic alterations in immune cells and tumor cells. The ability to simultaneously assess chromatin accessibility and V(D)J rearrangement in individual cells may yield insight into the complex interactions between tumor cells and immune cells in the TME tumor establishment and progression. This may lead to the discovery of valuable biomarkers for targeted therapy and a better understanding of the molecular mechanisms that underlie clinical response and resistance to immunomodulatory and epigenetic therapies.

Tagmentation of genomic DNA using a transposase loaded with adaptors with a 3′ single-stranded overhang on the non-transfer strand can be used for any application that depends on capture of genomic fragments by a complementary 3′ single-strand. Suitable transposases include, but are not limited to, prokaryotic transposases (e.g., Tn3, Tn5, Tn7, Tn10, phage Mu, etc., transposases) and eukaryotic transposases (e.g., Sleeping Beauty, PiggyBac, Hermes, etc., transposases). Preferred transposases include Tn5 transposases and functional mutants and derivatives thereof, including hyperactive Tn5 transposases, or a MuA transposase or a functional mutant or derivative thereof. The main application of the methods of the present disclosure is to allow to integrate a chromatin accessibility assay with existing scRNA-seq technologies that capture the 5′ end of transcripts, thus allowing capture of V(D)J rearrangement information of single cells. This enables simultaneous evaluation of transcription, clonality, and epigenetic state of single immune cells. Furthermore, the present platform also allows identification of antigen specificity alongside the modalities mentioned above as tetramer positive cells can be identified via the same modality as surface labeling.

Non-limiting applications of this technology include assessment of immune responses to cancer, infections, and vaccines. In each case, evaluation of all the available modalities—surface phenotype, transcriptional state, TCR or BCR identity/specificity, and chromatin accessibly—can enable determining what the lymphocytes are specific to, how they are responding (transcription), and what they are poised to become (memory, effector, etc.). This platform can enable insight into the immune landscape and may be used in cancer immunology and fundamental immunology research.

To facilitate an understanding of the principles and features of the various embodiments of the invention, various illustrative embodiments are explained below. Although exemplary embodiments of the invention are explained in detail, it is to be understood that other embodiments are contemplated. Accordingly, it is not intended that the invention is limited in its scope to the details of construction and arrangement of components set forth in the following description or examples. The invention is capable of other embodiments and of being practiced or carried out in various ways. Also, in describing the exemplary embodiments, specific terminology will be resorted to for the sake of clarity.

The term “about” or “approximately” means within a statistically meaningful range of a value. Such a range can be within an order of magnitude, preferably within 50%, more preferably within 20%, still more preferably within 10%, and even more preferably within 5% of a given value or range. The allowable variation encompassed by the term “about” or “approximately” depends on the particular system under study, and can be readily appreciated by one of ordinary skill in the art.

The terms “a,” “an,” and “the” do not denote a limitation of quantity, but rather denote the presence of “at least one” of the referenced item.

As used herein, the term “and/or” may mean “and,” it may mean “or,” it may mean “exclusive-or,” it may mean “one,” it may mean “some, but not all,” it may mean “neither,” and/or it may mean “both.” The term “or” is intended to mean an inclusive “or.”

Also, in describing the exemplary embodiments, terminology will be resorted to for the sake of clarity. It is intended that each term contemplates its broadest meaning as understood by those skilled in the art and includes all technical equivalents which operate in a similar manner to accomplish a similar purpose. It is to be understood that embodiments of the disclosed technology may be practiced without these specific details. In other instances, well-known methods, structures, and techniques have not been shown in detail in order not to obscure an understanding of this description. References to “one embodiment,” “an embodiment,” “example embodiment,” “some embodiments,” “certain embodiments,” “various embodiments,” etc., indicate that the embodiment(s) of the disclosed technology so described may include a particular feature, structure, or characteristic, but not every embodiment necessarily includes the particular feature, structure, or characteristic. Further, repeated use of the phrase “in one embodiment” does not necessarily refer to the same embodiment, although it may.

Throughout this disclosure, various aspects of the invention can be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. This applies regardless of the breadth of the range.

It is also to be understood that the mention of one or more method steps does not preclude the presence of additional method steps or intervening method steps between those steps expressly identified. Similarly, it is also to be understood that the mention of one or more components in a composition does not preclude the presence of additional components than those expressly identified.

The materials described hereinafter as making up the various elements of the present invention are intended to be illustrative and not restrictive. Many suitable materials that would perform the same or a similar function as the materials described herein are intended to be embraced within the scope of the invention. Such other materials not described herein can include, but are not limited to, materials that are developed after the time of the development of the invention, for example. Any dimensions listed in the various drawings are for illustrative purposes only and are not intended to be limiting. Other dimensions and proportions are contemplated and intended to be included within the scope of the invention.

### Methods of the Invention

In one aspect, the invention relates to a method for simultaneously measuring one or more of 5′ RNA transcripts, transcription profiles, chromatin accessibility, surface markers, and immune receptor sequence of a biological sample at the single-cell level, the sample comprising one or more cells.

In one aspect, the invention provides a method for tagmentation of genomic DNA in a cell, the method comprising: a) permeabilizing the cell; and b) incubating the permeabilized cell with a transposome assembly comprising a transposase, a first oligonucleotide, and a second oligonucleotide comprising a 3′ single-stranded overhang on the non-transfer strand, wherein the transposase makes double-strand breaks in genomic DNA and attaches adaptors at the ends of the genomic DNA fragments that are produced, thereby producing tagmented genomic DNA with a transposase recognition site and a complementary sequence to a capture sequence of a third oligonucleotide, the complementary sequence located at the single stranded 3′ end of the non-transfer strand of the tagmented genomic DNA.

In some embodiments, step (a) and step (b) are performed sequentially.

In some embodiments, step (a) and step (b) are performed simultaneously.

In some embodiments, the method further comprises generating the transposome assembly prior to step (b) by a method comprising: i) providing the first oligonucleotide comprising a free 5′ phosphate at a 5′ end of the first oligonucleotide and the transposase recognition site; ii) providing the second oligonucleotide comprising a free 5′ phosphate at a 5′ end of the second oligonucleotide, the transposase recognition site, and a sequence complementary to the capture sequence of the third oligonucleotide at the 3′ end of the second oligonucleotide; iii) providing a fourth oligonucleotide comprising a free 5′ phosphate at a 5′ end of the fourth oligonucleotide, the transposase recognition site, and a unique sequence at the 3′ end of the fourth oligonucleotide; iv) annealing the first oligonucleotide and second oligonucleotide to one another to form a first adaptor that contains a double-stranded transposase recognition site and a single-stranded 3′ overhang on a non-transfer strand, the single-stranded 3′ overhang comprising the sequence complementary to the capture sequence of the third oligonucleotide; iv) annealing the first oligonucleotide and fourth oligonucleotide to one another to form a second adaptor that contains a double-stranded transposase recognition site and a single-stranded 3′ overhang on a non-transfer strand, the single-stranded 3′ overhang comprising the unique sequence; and vi) incubating the first adaptor and the second adaptor with the transposase to form the transposome assembly. The unique sequence at the 3′ end of the fourth oligonucleotide can be used for downstream amplification and/or library preparation.

In some embodiments, the second oligonucleotide comprises a modification at the 3′ end to prevent degradation of the oligonucleotide, optionally wherein the modification is one or more phosphorothioate bonds.

In some embodiments, the transposase is a Tn5 transposase or a hyperactive Tn5 transposase or a functional mutant or derivative thereof, or a MuA transposase or a functional mutant or derivative thereof, and wherein the transposase recognition site is a Tn5 recognition site or a MuA recognition site comprising a R1 sequence and a R2 sequence.

In some embodiments, the transposase is a Tn5 transposase or a hyperactive Tn5 transposase or a functional mutant or derivative thereof, and wherein the transposase recognition site is the Tn5 recognition site.

In some embodiments, the method further comprises purifying the tagmented genomic DNA fragments.

In some embodiments, the method further comprises gap-filling the tagmented genomic DNA fragments.

In some embodiments, the gap-filling is performed by a non-hot start non-gap filling DNA polymerase.

In some embodiments, the method further comprises ligating the gap-filled tagmented genomic DNA fragments.

In some embodiments, the ligation is performed by a T4 DNA ligase or an E. coli DNA ligase.

In some embodiments, the method further comprises amplifying the ligated gap-filled tagmented genomic fragments DNA with primers.

In some embodiments, the method further comprises preparing an assay for transposase-accessible chromatin (ATAC) library from the amplified genomic DNA fragments.

In another aspect, the invention provides a method for simultaneously measuring the 5′ end of RNA transcripts and chromatin accessibility at the single-cell level in a biological sample comprising one or more cells, the method comprising: a) permeabilizing the cells in the biological sample; b) incubating the permeabilized cells with a plurality of transposome assemblies each comprising a transposase, a first adaptor, and a second adaptor comprising a 3′ single-stranded overhang on the non-transfer strand, wherein the transposase makes double-strand breaks in genomic DNA and attaches the first and second adaptors at the ends of the genomic DNA fragments that are produced, thereby producing form tagmented genomic DNA fragments flanked on both sides by a transposase recognition site and a sequence complementary to a capture sequence of a third oligonucleotide, the complementary sequence located at a single stranded 3′ end of the non-transfer strand of tagmented genomic DNA, or flanked on one side by a transposase recognition site and a sequence complementary to a capture sequence of a third oligonucleotide, the complementary sequence located at a single stranded 3′ end of the non-transfer strand of tagmented genomic DNA, and by a transposase recognition site and a unique sequence on the other side; c) incubating the permeabilized cells with a carrier comprising the third oligonucleotide comprising one or more of a barcode sequence, a cell barcode sequence, and a unique molecular identifier (UMI), and d) separating individual cells, each separated cell comprising the carrier comprising the third oligonucleotide, mRNA molecules, and tagmented genomic DNA fragments; e) generating cDNA from the mRNA molecules, the cDNA being captured by the third oligonucleotide attached to the carrier; f) purifying the cDNA and tagmented genomic DNA fragments; g) performing gap-filling on the tagmented genomic DNA fragments; h) ligating to seal nicks in the gap-filled tagmented genomic DNA fragments; i) amplifying the cDNA and the ligated gap-filled tagmented genomic DNA fragments with primers and separating the amplified cDNA from the amplified genomic DNA fragments; j) preparing a cDNA library from the amplified cDNA; and k) preparing an assay for transposase-accessible chromatin (ATAC) library from the amplified genomic DNA fragments.

In some embodiments, the transposase is a Tn5 transposase or a hyperactive Tn5 transposase or a functional mutant or derivative of any said transposase, or a MuA transposase or a functional mutant or derivative thereof, and wherein the transposase recognition site is a Tn5 recognition site or a MuA recognition site comprising a R1 sequence and a R2 sequence.

In some embodiments, the transposase is a Tn5 transposase or a hyperactive Tn5 transposase or a functional mutant or derivative thereof, and wherein the transposase recognition site is the Tn5 recognition site.

In some embodiments, step (a) and step (b) are performed sequentially.

In some embodiments, step (a) and step (b) are performed simultaneously.

In some embodiments, step (g) and step (h) are performed sequentially.

In some embodiments, step (g) and step (h) are performed simultaneously.

In some embodiments, the method comprises generating the transposome assembly comprising the transposase, the first adaptor, and the second adaptor prior to step (b).

In some embodiments, generating the transposome assembly comprises: providing a first oligonucleotide comprising a free 5′ phosphate at a 5′ end of the first oligonucleotide and the transposase recognition site; providing a second oligonucleotide comprising a free 5′ phosphate at a 5′ end of the second oligonucleotide, the transposase recognition site, and the sequence complementary to the capture sequence of the third oligonucleotide at the 3′ end of the second oligonucleotide; providing a fourth oligonucleotide comprising a free 5′ phosphate at a 5′ end of the fourth oligonucleotide, the transposase recognition site, and a unique sequence at the 3′ end of the fourth oligonucleotide; annealing the first oligonucleotide and second oligonucleotide to one another to form the first adaptor that contains a double-stranded transposase recognition site and a single-stranded 3′ overhang on a non-transfer strand, the single-stranded 3′ overhang comprising the sequence complementary to the capture sequence of the third oligonucleotide; annealing the first oligonucleotide and fourth oligonucleotide to one another to form the second adaptor that contains a double-stranded transposase recognition site and a single-stranded 3′ overhang on a non-transfer strand, the single-stranded 3′ overhang comprising the unique sequence; and incubating the first adaptor and the second adaptor with the transposase to form the transposome assembly. The unique sequence at the 3′ end of the fourth oligonucleotide can be used for downstream amplification and/or library preparation.

In some embodiments, the second adaptor comprises a modification at the 3′ end to prevent degradation of the oligonucleotide, optionally wherein the modification is one or more phosphorothioate bonds.

In some embodiments, the carrier is a bead or a solid surface.

In some embodiments, the method further comprises prior to step (a), labeling the cells in the biological sample with oligonucleotide-labeled lipids, oligonucleotide-tagged hashing antibodies, or oligonucleotide-tagged antibodies against specific surface markers.

In some embodiments, the cells are labeled with the oligonucleotide-labeled lipids, the oligonucleotide-tagged hashing antibodies, or the oligonucleotide-tagged antibodies against specific surface markers, the method comprising: generating antibody-derived tag (ADT) fragments from the oligonucleotide-tagged antibodies, the ADT fragments being captured by the third oligonucleotide attached to the carrier; purifying the ADT fragments; separating the ADT fragments from the amplified genomic DNA fragments and/or cDNA; amplifying the ADT fragments; and preparing an ADT library from the amplified ADT fragments.

In some embodiments, non-limiting exemplary cell surface markers include CD298, 32 microglobulin, CD45, and MHC class I molecules.

In some embodiments, step (a) further comprises incubation with a RNase inhibitor.

In some embodiments, step (c) further comprises counting the number of cells in the biological sample, resuspending the cells at a concentration of 150-1500 cells/μl, and processing the resuspended cells for bead-based single-cell RNA-sequencing.

In some embodiments, step (b) further comprises inactivating the transposase.

In some embodiments, generating cDNA in step (e) comprises: reverse transcribing the mRNA with a reverse transcriptase enzyme to generate the cDNA fragments; and inactivating the reverse transcriptase enzyme.

In some embodiments, the transposase is inactivated by heating to 65° C., incubating with 0.04% SDS at room temperature, or by the addition of EDTA.

In some embodiments, step (f) comprises forming a plurality of droplets, each droplet comprising an individual cell.

In some embodiments, the gap-filling in step (g) is performed by a non-hot start non-gap filling DNA polymerase.

In some embodiments, the ligation in step (h) is performed by a T4 DNA ligase or an E. coli DNA ligase.

In some embodiments, the cDNA is used to amplify a V(D)J region of transcripts using specific primers to generate a library of V(D)J fragments.

In some embodiments, the cDNA library, ADT library, V(D)J library, and ATAC library are pooled after they are generated.

In some embodiments, the cDNA library, ADT library, V(D)J library, and ATAC library are sequenced in parallel.

In some embodiments, the biological sample is characterized based upon the sequencing readout.

In some embodiments, the biological sample comprises a single cell, and the single readout comprises a single cell readout.

In some embodiments, the method further comprises associating a cDNA fragment, an ADT fragment, a V(D)J fragment, and/or genomic DNA fragment with a single cell in the biological sample based on the cell barcode sequence of third oligonucleotide.

In some embodiments, after a sequencing analysis, the biological sample is assigned to a sample of origin based on a barcode sequence of the carrier and/or of an oligonucleotide-tagged antibody, an oligonucleotide-tagged hashing antibody, or an oligonucleotide-tagged lipid in the ADT library.

In some embodiments, the method further comprises analyzing the sequencing analysis to generate a representation of accessible chromatin, a transcriptomic profile, an immune receptor repertoire, and/or a surface epitope repertoire of a single cell in the biological sample.

In another aspect, the invention provides a method for simultaneously measuring the 5′ end of transcripts, chromatin accessibility, surface markers, and immune receptor sequence of a biological sample comprising one or more cells at the single-cell level, the method comprising: a) labeling cells in the biological sample with oligonucleotide-tagged lipids, oligonucleotide-tagged hashing antibodies, or oligonucleotide-tagged antibodies against specific surface markers; b) permeabilizing the cells in the biological sample; c) incubating permeabilized cells with a plurality of transposome assemblies each comprising a transposase, a first adaptor, and a second adaptor comprising a 3′ single-stranded overhang on the non-transfer strand, wherein the transposase makes double-strand breaks in genomic DNA and attaches the first and second adaptors at the ends of the genomic DNA fragments that are produced, thereby producing tagmented genomic DNA fragments flanked on both sides by a transposase recognition site and a sequence complementary to a capture sequence of a third oligonucleotide, the complementary sequence located at a single stranded 3′ end of the non-transfer strand of tagmented genomic DNA fragments, or flanked on one side by a transposase recognition site and a sequence complementary to a capture sequence of a third oligonucleotide, the complementary sequence located at a single stranded 3′ end of the non-transfer strand of tagmented genomic DNA, and by a transposase recognition site and a unique sequence on the other side; d) incubating the permeabilized cells with a carrier comprising the third oligonucleotide containing one or more of a barcode sequence, a cell barcode sequence, and a unique molecular identifier (UMI); e) separating individual cells, each separated cell comprising the carrier comprising the third oligonucleotide, mRNA molecules, tagmented genomic DNA fragments, and oligonucleotide-tagged antibodies, oligonucleotide-tagged hashing antibodies, or oligonucleotide-tagged lipids from a single cell; f) generating cDNA from the mRNA molecules and generating ADT fragments from the oligonucleotide-tagged antibodies, oligonucleotide-tagged hashing antibodies, or the oligonucleotide-tagged lipids, the cDNA and ADT fragments being captured by the third oligonucleotide attached to the carrier; g) purifying the cDNA, the ADT fragments, and the tagmented genomic DNA fragments; h) performing gap-filling on the tagmented genomic DNA fragments; i) ligating to seal nicks in the gap-filled tagmented genomic DNA fragments; j) amplifying the cDNA, the ADT fragments, and the ligated gap-filled tagmented genomic DNA fragments with primers and separating each of the amplified cDNA, the amplified ADT fragments, and the amplified genomic DNA fragments; k) preparing a cDNA library from the amplified cDNA; 1) preparing an ADT library from the amplified ADT fragments; and m) preparing an ATAC library from the amplified genomic DNA fragments.

In some embodiments, the transposase is a Tn5 transposase or a hyperactive Tn5 transposase or a functional mutant or derivative of any said transposase, or a MuA transposase or a functional mutant or derivative thereof, and wherein the transposase recognition site is a Tn5 recognition site or a MuA recognition site comprising a R1 sequence and a R2 sequence.

In some embodiments, the transposase is a Tn5 transposase or a hyperactive Tn5 transposase or a functional mutant or derivative of any said transposase, and wherein the transposase recognition site is a Tn5 recognition site.

In some embodiments, step (a) and step (b) are performed sequentially.

In some embodiments, step (a) and step (b) are performed simultaneously.

In some embodiments, step (g) and step (h) are performed sequentially.

In some embodiments, step (g) and step (h) are performed simultaneously.

In some embodiments, the method comprises generating the transposome assembly comprising the transposase, the first adaptor, and the second adaptor prior to step (b).

In some embodiments, generating the transposome assembly comprises: providing a first oligonucleotide comprising a free 5′ phosphate at a 5′ end of the first oligonucleotide and the transposase recognition site; providing a second oligonucleotide comprising a free 5′ phosphate at a 5′ end of the second oligonucleotide, the transposase recognition site, and the sequence complementary to the capture sequence of the third oligonucleotide at the 3′ end of the second oligonucleotide; providing a fourth oligonucleotide comprising a free 5′ phosphate at a 5′ end of the fourth oligonucleotide, the transposase recognition site, and a unique sequence at the 3′ end of the fourth oligonucleotide; annealing the first oligonucleotide and second oligonucleotide to one another to form the first adaptor that contains a double-stranded transposase recognition site and a single-stranded 3′ overhang on a non-transfer strand, the single-stranded 3′ overhang comprising the sequence complementary to the capture sequence of the third oligonucleotide; annealing the first oligonucleotide and fourth oligonucleotide to one another to form the second adaptor that contains a double-stranded transposase recognition site and a single-stranded 3′ overhang on a non-transfer strand, the single-stranded 3′ overhang comprising the unique sequence; and incubating the first adaptor and the second adaptor with the transposase to form the transposome assembly. The unique sequence at the 3′ end of the fourth oligonucleotide can be used for downstream amplification and/or library preparation.

In some embodiments, the second oligonucleotide comprises a modification at the 3′ end to prevent degradation of the oligonucleotide, optionally wherein the modification is one or more phosphorothioate bonds.

In some embodiments, the carrier is a bead or a solid surface.

In some embodiments, step (a) further comprises incubation with a RNase inhibitor.

In some embodiments, step (c) further comprises counting the number of cells in the biological sample, resuspending the cells at a concentration of 150-1500 cells/μl, and processing the resuspended cells for bead-based single-cell RNA-sequencing.

In some embodiments, step (b) further comprises inactivating the transposase.

In some embodiments, generating cDNA in step (e) comprises: reverse transcribing the mRNA with a reverse transcriptase enzyme to generate the cDNA fragments; and inactivating the reverse transcriptase enzyme.

In some embodiments, the transposase is inactivated by heating to 65° C., incubating with 0.04% SDS at room temperature, or by the addition of EDTA.

In some embodiments, step (f) comprises forming a plurality of droplets, each droplet comprising an individual cell.

In some embodiments, the gap-filling in step (g) is performed by a non-hot start non-gap filling DNA polymerase.

In some embodiments, the ligation in step (h) is performed by a T4 DNA ligase or an E. coli DNA ligase.

In some embodiments, the cDNA is used to amplify a V(D)J region of transcripts using specific primers to generate a library of V(D)J fragments.

In some embodiments, the cDNA library, ADT library, V(D)J library, and ATAC library are pooled after they are generated.

In some embodiments, the cDNA library, ADT library, V(D)J library, and ATAC library are sequenced in parallel.

In some embodiments, the biological sample is characterized based upon the sequencing readout.

In some embodiments, the biological sample comprises a single cell, and the single readout comprises a single cell readout.

In some embodiments, the method further comprises associating a cDNA fragment, an ADT fragment, a V(D)J fragment, and/or genomic DNA fragment with a single cell in the biological sample based on the cell barcode sequence of third oligonucleotide.

In some embodiments, after a sequencing analysis, the biological sample is assigned to a sample of origin based on a barcode sequence of the carrier and/or of an oligonucleotide-tagged antibody, an oligonucleotide-tagged hashing antibody, or an oligonucleotide-tagged lipid in the ADT library.

In some embodiments, the method further comprises analyzing the sequencing analysis to generate a representation of accessible chromatin, a transcriptomic profile, an immune receptor repertoire, and/or a surface epitope repertoire of a single cell in the biological sample.

In an aspect, the invention provides a method for processing a sample, the method comprising: a) contacting a plurality of permeabilized cells labeled with oligonucleotide-tagged antibodies, oligonucleotide-tagged hashing antibodies, or oligonucleotide-tagged lipids with a plurality of transposome assemblies to generate cells comprising a plurality of tagged genomic DNA fragments, wherein the tag is located at a single stranded 3′overhang end of the non-transfer strand of each of the plurality of the tagged genomic DNA fragments; b) generating cDNA from mRNA; c) generating a plurality of antibody-derived tag (ADT) fragments from the oligonucleotide-tagged antibodies, oligonucleotide-tagged hashing antibodies, or the oligonucleotide-tagged lipids; d) partitioning the plurality of permeabilized cells and a carrier comprising a plurality of barcode sequences into a plurality of partitions, wherein at least one of the plurality of partitions comprises (i) one of plurality of permeabilized cells comprising the plurality of tagged genomic DNA fragments, the cDNA, and the plurality of ADT fragments; and (ii) one of the plurality of beads, wherein the carrier comprises a barcode oligonucleotide comprising a first barcode sequence and a second barcode sequence; and e) generating: (i) a first barcoded molecule comprising (1) a sequence of the plurality of tagged genomic DNA fragment, and (2) the barcode oligonucleotide, or a reverse complement thereof; and (ii) a second barcoded molecule comprising (1) a sequence of the cDNA or of a sequence of the plurality of the ADT fragments, and (2) the barcode oligonucleotide, or a reverse complement thereof.

In some embodiments, step (a) comprises: providing a transposase that makes double-strand breaks in genomic DNA and attaches adaptors at the ends of the genomic DNA fragments that are produced; providing a first oligonucleotide comprising a free 5′ phosphate at a 5′ end of the first oligonucleotide and a transposase recognition site; providing a second oligonucleotide comprising a free 5′ phosphate at a 5′ end of the second oligonucleotide, the transposase recognition site, and the sequence complementary to the barcode oligonucleotide at a 3′ overhang end of the second oligonucleotide; annealing the first oligonucleotide and second oligonucleotide to one another to form an adaptor that contains a double-stranded transposase recognition site and a single-stranded 3′ overhang on a non-transfer strand, the single-stranded 3′ overhang comprising the sequence complementary to the barcode oligonucleotide; and incubating the adaptor with the transposase to form the transposome assembly.

In some embodiments, the transposase is a Tn5 transposase or a hyperactive Tn5 transposase or a functional mutant or derivative of any of said transposases, or a MuA transposase or a functional mutant or derivative thereof, and wherein the transposase recognition site is a Tn5 recognition site or a MuA recognition site comprising a R1 sequence and a R2 sequence.

In some embodiments, the transposase is a Tn5 transposase or a hyperactive Tn5 transposase or a functional mutant or derivative of any of said transposases, and wherein the transposase recognition site is a Tn5 recognition site.

In some embodiments, the second oligonucleotide comprises a modification at the 3′ end to prevent degradation of the oligonucleotide, optionally wherein the modification is one or more phosphorothioate bonds.

In some embodiments, step (a) or step (b) further comprises incubation with a RNase inhibitor.

In some embodiments, step (d) further comprises counting the number of cells in the biological sample, resuspending at a concentration of 150-1500 cells/μl, and processing the resuspended biological sample for bead-based single-cell RNA-sequencing.

In some embodiments, step (b) further comprises inactivating the transposase.

In some embodiments, step (b) further comprises: (i) reverse transcribing the mRNA present in the biological sample with a reverse transcriptase enzyme to generate the cDNA fragments and ADT fragments; and (ii) inactivating the reverse transcriptase enzyme.

In some embodiments, the transposase is inactivated by heating to 65° C., incubating with 0.04% SDS at room temperature, or by the addition of EDTA.

In some embodiments, the carrier comprises a bead or a solid surface.

In some embodiments, gap filling using a non-hot start non-strand-displacing DNA polymerase is performed after step (b).

In some embodiments, ligation to seal the nicks in the gap-filled tagmented genomic DNA fragments is performed after the gap filling.

In some embodiments, the ligation is performed by T4 DNA ligase or by E. coli DNA ligase.

In some embodiments, the method further comprises sequencing (i) the first barcoded molecule or a derivative generated from the first barcoded molecule, and (ii) the second barcoded molecule or a derivative generated from the second barcoded molecule to generate a plurality of sequencing reads corresponding to the of genomic DNA fragment, the ADT fragment, and the cDNA.

In some embodiments, the cDNA is used to amplify a V(D)J region of transcripts using specific primers to generate a plurality of V(D)J fragments.

In some embodiments, the cDNA, plurality of ADT fragments, plurality of V(D)J fragments, and plurality of tagged genomic DNA fragments are pooled after they are generated.

In some embodiments, the plurality of cDNA fragments, plurality of ADT fragments, plurality of V(D)J fragments, and plurality of tagged genomic DNA fragments are sequenced in parallel.

In some embodiments, the biological sample is characterized based upon the sequencing readout.

In some embodiments, the biological sample comprises a single cell, and the single readout comprises a single cell readout.

In some embodiments, the method further comprises associating a cDNA fragment, an ADT fragment, a V(D)J fragment, and/or a tagged genomic DNA fragment with a single cell in the biological sample based on the cell barcode sequence of third oligonucleotide.

In some embodiments, the method further comprises associating the tagged fragment of genomic DNA, the cDNA fragment, the ADT fragment, and/or the V(D)J fragment with the permeabilized cell based on the sequencing reads.

In some embodiments, the method further comprises analyzing the sequencing reads to generate a representation of one or more of chromatin accessibility, transcriptome profile, surface markers, and immune cell repertoire of the permeabilized cell.

In some embodiments, one or more of the partitions comprises at most a single cell of the plurality of cells.

In some embodiments, one or more of the partitions comprises at most a single bead of the plurality of beads.

In an aspect, the invention provides a method for generating barcoded nucleic acid fragments, the method comprising: a) permeabilizing a cell; b) incubating the permeabilized cell with a plurality of transposome assemblies comprising a transposase, a first adaptor, and a second adaptor comprising a 3′ single-stranded overhang on the non-transfer strand, wherein the transposase makes double-strand breaks in genomic DNA and attaches the first and second adaptors at the ends of the genomic DNA fragments that are produced, thereby producing tagmented genomic DNA fragments flanked on both sides by a transposase recognition site and a sequence complementary to a capture sequence of a third oligonucleotide, the complementary sequence located at a single stranded 3′ end of the non-transfer strand of tagmented genomic DNA fragments, or flanked on one side by a transposase recognition site and a sequence complementary to a capture sequence of a third oligonucleotide, the complementary sequence located at a single stranded 3′ end of the non-transfer strand of tagmented genomic DNA, and by a transposase recognition site and a unique sequence on the other side; c) generating cDNA from mRNA; d) purifying the cDNA and tagmented genomic DNA fragments; e) performing gap-filling on the tagmented genomic DNA fragments; f) ligating to seal nicks in the gap-filled tagmented genomic DNA fragments; g) amplifying the cDNA and tagmented genomic DNA fragments with primers and separating the amplified cDNA from the amplified genomic DNA fragments; h) preparing a cDNA library from the amplified cDNA; i) preparing an ATAC library from the amplified genomic DNA fragments; and j) incubating each of the cDNA library and the ATAC library with a carrier comprising the third oligonucleotide to form a complex comprising the carrier and a member of the cDNA library or a member of the ATAC library.

In some embodiments, the transposase is a Tn5 transposase or a hyperactive Tn5 transposase or a functional mutant or derivative of any said transposase, or a MuA transposase or a functional mutant or derivative thereof, and wherein the transposase recognition site is a Tn5 recognition site or a MuA recognition site comprising a R1 sequence and a R2 sequence.

In some embodiments, the transposase is a Tn5 transposase or a hyperactive Tn5 transposase or a functional mutant or derivative thereof, and wherein the transposase recognition site is the Tn5 recognition site.

In some embodiments, step (a) and step (b) are performed sequentially.

In some embodiments, step (a) and step (b) are performed simultaneously.

In some embodiments, step (e) and step (f) are performed sequentially.

In some embodiments, step (e) and step (f) are performed simultaneously.

In some embodiments, the method comprises generating the transposome assembly comprising the transposase, the first adaptor, and the second adaptor prior to step (b).

In some embodiments, generating the transposome assembly comprises: providing a first oligonucleotide comprising a free 5′ phosphate at a 5′ end of the first oligonucleotide and the transposase recognition site; providing a second oligonucleotide comprising a free 5′ phosphate at a 5′ end of the second oligonucleotide, the transposase recognition site, and the sequence complementary to the capture sequence of the third oligonucleotide at the 3′ end of the second oligonucleotide; providing a fourth oligonucleotide comprising a free 5′ phosphate at a 5′ end of the fourth oligonucleotide, the transposase recognition site, and a unique sequence at the 3′ end of the fourth oligonucleotide; annealing the first oligonucleotide and second oligonucleotide to one another to form the first adaptor that contains a double-stranded transposase recognition site and a single-stranded 3′ overhang on a non-transfer strand, the single-stranded 3′ overhang comprising the sequence complementary to the capture sequence of the third oligonucleotide; annealing the first oligonucleotide and fourth oligonucleotide to one another to form the second adaptor that contains a double-stranded transposase recognition site and a single-stranded 3′ overhang on a non-transfer strand, the single-stranded 3′ overhang comprising the unique sequence; and incubating the first adaptor and the second adaptor with the transposase to form the transposome assembly. The unique sequence at the 3′ end of the fourth oligonucleotide can be used for downstream amplification and/or library preparation.

In some embodiments, the second oligonucleotide comprises a modification at the 3′ end to prevent degradation of the oligonucleotide, optionally wherein the modification is one or more phosphorothioate bonds.

In some embodiments, the carrier is a bead or a solid surface.

In some embodiments, the method further comprises prior to step (a), labeling the cell with oligonucleotide-labeled lipids, oligonucleotide-tagged hashing antibodies, or oligonucleotide-tagged antibodies against specific surface markers.

In some embodiments, the cell is labeled with the oligonucleotide-labeled lipids, the oligonucleotide-tagged hashing antibodies, or the oligonucleotide-tagged antibodies or against specific surface markers, the method comprising: generating antibody-derived tag (ADT) fragments from the oligonucleotide-tagged antibodies or the oligonucleotide-tagged hashing antibodies, the ADT fragments being captured by the third oligonucleotide attached to the carrier; purifying the ADT fragments; separating the ADT fragments from the amplified genomic DNA fragments and/or cDNA; amplifying the ADT fragments; and preparing an ADT library from the amplified ADT fragments.

In some embodiments, non-limiting exemplary cell surface markers include CD298, 32 microglobulin, CD45, and MHC class I molecules.

In some embodiments, step (a) further comprises incubation with a RNase inhibitor.

In some embodiments, step (b) further comprises inactivating the transposase.

In some embodiments, generating cDNA in step (c) comprises: reverse transcribing the mRNA with a reverse transcriptase enzyme to generate the cDNA fragments; and inactivating the reverse transcriptase enzyme.

In some embodiments, the transposase is inactivated by heating to 65° C., incubating with 0.04% SDS at room temperature, or by the addition of EDTA.

In some embodiments, the gap-filling in step (e) is performed by a non-hot start non-gap filling DNA polymerase.

In some embodiments, the ligation in step (f) is performed by a T4 DNA ligase or an E. coli DNA ligase.

In some embodiments, the cDNA is used to amplify a V(D)J region of transcripts using specific primers to generate a library of V(D)J fragments.

In some embodiments, the cDNA library, ADT library, V(D)J library, and ATAC library are pooled after they are generated.

In some embodiments, the cDNA library, ADT library, V(D)J library, and ATAC library are sequenced in parallel.

In some embodiments, the biological sample is characterized based upon the sequencing readout.

In some embodiments, after a sequencing analysis, the biological sample is assigned to a sample of origin based on a barcode sequence of the carrier and/or of an oligonucleotide-tagged antibody, an oligonucleotide-tagged hashing antibodies, or an oligonucleotide-tagged lipid in the ADT library.

In some embodiments, the method further comprises analyzing the sequencing analysis to generate a representation of accessible chromatin, a transcriptomic profile, an immune receptor repertoire, and/or a surface epitope repertoire of the single cell.

In an aspect, the invention provides a method for processing a sample, the method comprising: a) contacting a plurality of permeabilized cells with a transposome assembly comprising a transposase, a first oligonucleotide, and a second oligonucleotide comprising a 3′ single-stranded overhang on the non-transfer strand, thereby producing tagmented genomic DNA fragments with a transposase recognition site and a complementary sequence to a capture sequence of a third oligonucleotide, the complementary sequence located at the single stranded 3′ end of the non-transfer strand of the tagmented genomic DNA fragments; b) generating cDNA from mRNA; and c) optionally generating: (i) a first barcoded molecule comprising (1) a sequence of the tagged fragment of genomic DNA, and (2) a first barcode, or a reverse complement thereof, the first barcode or reverse complement attached to a bead; and (ii) a second barcoded molecule comprising (1) a sequence of the cDNA fragment, and (2) a second barcode, or a reverse complement thereof, the second barcode or reverse complement attached to the bead.

In some embodiments, step (a) comprises: providing a transposase that makes double-strand breaks in genomic DNA and attaches adaptors at the ends of the genomic DNA fragments that are produced; providing a first oligonucleotide comprising a free 5′ phosphate at a 5′ end of the first oligonucleotide and a transposase recognition site; providing a second oligonucleotide comprising a free 5′ phosphate at a 5′ end of the second oligonucleotide, the transposase recognition site, and a sequence complementary to the first barcode oligonucleotide at a 3′ end of the second oligonucleotide; annealing the first oligonucleotide and second oligonucleotide to one another to form an adaptor that contains a double-stranded transposase recognition site and a single-stranded 3′ overhang on a non-transfer strand, the single-stranded 3′ overhang comprising the sequence complementary to the first barcode oligonucleotide; and incubating the plurality of adaptors with a transposase to form the transposome assembly.

In some embodiments, the second oligonucleotide comprises a modification at the 3′ end to prevent degradation of the oligonucleotide, optionally wherein the modification is one or more phosphorothioate bonds.

In some embodiments, the transposase is a Tn5 transposase or a hyperactive Tn5 transposase or a functional mutant or derivative of any of said transposases, or a MuA transposase or a functional mutant or derivative thereof, and wherein the transposase recognition site is a Tn5 recognition site or a MuA recognition site comprising a R1 sequence and a R2 sequence.

In some embodiments, the transposase is a Tn5 transposase or a hyperactive Tn5 transposase or a functional mutant or derivative of any of said transposases, and wherein the transposase recognition site is a Tn5 recognition site.

In some embodiments, step (b) further comprises incubation with a RNase inhibitor.

In some embodiments, step (a) further comprises counting the cells in the biological sample, resuspending at a concentration of 150-1500 cells/μl, and processing the resuspended biological sample for bead-based single-cell RNA-sequencing before generating the cDNA.

In some embodiments, step (b) further comprises inactivating the transposase.

In some embodiments, step (b) further comprises: reverse transcribing the mRNA present in the biological sample with a reverse transcriptase enzyme to generate the cDNA; and inactivating the reverse transcriptase enzyme.

In some embodiments, the transposase is inactivated by heating to 65° C., incubating with 0.04% SDS at room temperature, or by the addition of EDTA.

In some embodiments, gap filling using a non-hot start non-strand-displacing DNA polymerase is performed after step (b).

In some embodiments, ligation to seal the nicks in the cDNA fragments and tagmented genomic DNA using a ligase.

In some embodiments, the ligase is T4 DNA ligase or E. coli DNA ligase.

In some embodiments, the method further comprises sequencing (i) the first barcoded molecule or a derivative generated from the first barcoded molecule, and (ii) the second barcoded molecule or a derivative generated from the second barcoded molecule to generate a plurality of sequencing reads corresponding to the tagged fragment of genomic DNA and the cDNA.

In some embodiments, the method further comprises associating the tagged fragment of genomic DNA and the cDNA fragment with the permeabilized cell based on the sequencing reads.

In some embodiments, the method further comprises analyzing the sequencing reads to generate a representation of one or more of accessible chromatin, transcriptome profile, and immune cell repertoire of the permeabilized cell.

In some embodiments, one or more of the partitions comprises at most a single cell of the plurality of cells.

In some embodiments, one or more of the partitions comprises at most a single bead of the plurality of beads.

### Permeabilizing Cells

In one aspect, the method comprises permeabilizing the cells present in the biological sample in order to measure the modalities described herein. In some embodiments, the permeabilization is performed in a buffer comprising digitonin. In some embodiments, the digitonin is present in an amount of 0.01% to 0.5%. In some embodiments, the digitonin is present in an amount of 0.01% to 0.15%.

### Tagmenting Genomic DNA

In one aspect, the method comprises tagmentation of genomic DNA in the permeabilized cells and generating a library of tagmented genomic DNA fragments to determine chromatin accessibility.

In some embodiments, tagmentation is performed by a transposase. Suitable transposases include, but are not limited to, prokaryotic transposases (e.g., Staphylococcus aureus Tn552, Ty1, Tn/O and IS10, Tn3, Tn5, Tn7, Tn10, and phage MuA transposases) and eukaryotic transposases (e.g., Sleeping Beauty, PiggyBac, Hermes, and Mariner transposases). Preferred transposases include Tn5 transposases or functional mutants or derivatives thereof, including hyperactive Tn5 transposases, and MuA transposases or functional mutants or derivatives thereof. Some embodiments comprise a hyperactive Tn5 transposase and a Tn5-type transposase recognition site, or a MuA transposase and a Mu transposase recognition site comprising a R1 and R2 end sequence. Transposases suitable for use in the methods described herein should be capable of making a double-stranded break in genomic DNA and in the process attach adaptors at the ends of the fragment that is produced.

Most of the transposases possess no or very low transpositional activity in their native form or require a multimeric complex for their action, making it difficult or impossible to use them as efficient tools currently. Others, such as Tn5, MuA, PiggyBac, and Sleeping Beauty, are able to transpose without the need for auxiliary proteins. Transposition systems based on Tn5 or MuA transposases are used in many biotechnological applications.

In preferred embodiments, the method uses hyperactive Tn5 transposase because it is the most widely used, its recognition sequence is part of the sequence of the Illumina Nextera Read 2 sequencing primer, making it easier to integrate into the standard workflow, and Tn5 is commercially available. However, other transposases can be adapted for the application. More examples of transposition systems that may be used include Staphylococcus aureus Tn552 (Rowland, 1990), Ty1 (Devine, 1996), Transposon Tn7 (Craig,1996), Tn/O and IS10 (Kleckner, 1996), Mariner transposase (Lampe, 1996), Tc1 (Plasterk, 1996), Tn3 (Ichikawa,1990).

In some embodiments, tagmentation comprises generating a transposome assembly comprising a transposase, a first oligonucleotide, and a second oligonucleotide, and thereby producing tagmented genomic DNA comprising a transposase recognition site and a complementary sequence to a capture sequence of a third oligonucleotide, wherein the complementary sequence is located at a single stranded 3′ end on a non-transfer strand of the tagmented genomic DNA.

In some embodiments, the generation of the transposome assembly comprises: providing the first oligonucleotide comprising a free 5′ phosphate at a 5′ end of the first oligonucleotide and the transposase recognition site; providing the second oligonucleotide comprising a free 5′ phosphate at a 5′ end of the second oligonucleotide, the transposase recognition site, and the sequence complementary to the capture sequence of the third oligonucleotide at a 3′ end of the second oligonucleotide; annealing the first oligonucleotide and second oligonucleotide to one another to form a plurality of adaptors that contain a double-stranded ME sequence and a single-stranded 3′ overhang on a non-transfer strand, the single-stranded 3′ overhang comprising the sequence complementary to the capture sequence of the third oligonucleotide; and incubating the plurality of adaptors with the transposase to form the transposome assembly.

In some embodiments, the second oligonucleotide comprises a modification at the 3′ end to prevent degradation of the oligonucleotide, optionally wherein the modification is one or more phosphorothioate bonds.

In some embodiments, the transposome assembly forms tagmented genomic DNA fragments flanked by a mosaic end (ME) sequence and a sequence complementary to a capture sequence of a third oligonucleotide, the complementary sequence located at a single stranded 3′ end of the non-transfer strand of tagmented genomic DNA.

In some embodiments, a carrier (e.g., a bead or a solid surface) comprises the third oligonucleotide, the third oligonucleotide comprising (i) the capture sequence complementary to the sequence on the tagmented genomic DNA and (ii) at least one of a barcode sequence, a cell barcode sequence, and/or a unique molecular identifier (UMI).

In some embodiments, the transposase is inactivated. In some embodiments, the inactivation comprises incubating the tagmented genomic DNA fragments for 5 minutes at room temperature to remove the transposase from the DNA. In some embodiments, the inactivation comprises heating to 65° C., incubating with 0.04% SDS at room temperature, or addition of EDTA.

In some embodiments, gap-filling is performed on the purified tagmented genomic DNA fragments. In some embodiments, the gap-filling is performed by a DNA polymerase, such as for example and not limitation, a non-hot start non-strand-displacing DNA polymerase. In some embodiments, the DNA polymerase is KAPA HiFi non-hot start DNA polymerase or T4 DNA polymerase. In some embodiments, the gap-filling is performed at 12° C. for 15 minutes by T4 DNA polymerase or at 37 degrees for 10 minutes by KAPA HiFi non-hot start enzyme and buffer.

In some embodiments, the gap-filled tagmented genomic DNA fragments are ligated. In some embodiments, the ligation is performed by a DNA ligase, such as for example and not limitation, T3 ligase, T4 ligase, T7 ligase, Taq ligase, and E. coli ligase. In some embodiments using the E. coli ligase, the ligation is performed at 16° C. for 30 minutes followed by inactivation of the E. coli ligase at 65° C. for 20 minutes. In some embodiments using T7 DNA ligase, T4 ligase buffer can be used and the ligation is performed at room temperature for 15 minutes. In some embodiments, the gap-filling is performed before the ligation. In some embodiments, the gap-filling and ligation are performed simultaneously, e.g., by incubating the tagmented genomic DNA fragments with T4 DNA polymerase and Ampligase at 30° C. for 30 minutes or E. coli ligase at 16° C. for 30 minutes.

In some embodiments, the ligated genomic DNA fragments are amplified with primers. In some embodiments, the amplified genomic DNA fragments are used to generate a library by using indexing primers containing appropriate identifying sequences for use in a sequencer.

Generating a cDNA and/or Antibody-Derived Tag (ADT) Library

In one aspect, the method comprises generating a library of cDNA fragments from the mRNA of the permeabilized cells to provide a profile of 5′ RNA transcripts and of immune receptor sequences. The specific primers described herein can be used to focus amplification on specific families of V(D)J rearrangements and/or isotypes in order to provide a surface marker profile of an individual cell.

In some embodiments, the mRNA is reverse transcribed by a reverse transcriptase. In some embodiments, the antibody-derived tag (ADT) fragments are reverse transcribed by the reverse transcriptase. In some embodiments, the reverse transcription is performed at 53° C. for 45 minutes and at 85° C. for 5 minutes.

In some embodiments, the reverse transcriptase is inactivated.

### Characterizing Cell Surface Markers

In an aspect, the method comprises labeling and profiling of cell surface markers of the permeabilized cells, such as for example and not limitation, B cells and T cells as well as characterizing the markers, such as for example and not limitation, B cell receptors (BCRs) and T cell receptors (TCRs). Labeling the cell surface markers can comprise the use of oligonucleotide-tagged antibodies, oligonucleotide-tagged hashing antibodies, and/or oligonucleotide-tagged lipids. In the case of B cells, the method can be used to identify isotypes of IgH and IgL present on the surface of B cells. In the case of T cells, the method can be used to identify isotypes of TCR alpha, beta, gamma, and delta present on the surface of T cells.

In some embodiments, labeling the cell surface markers comprises the use of oligonucleotide-labeled hashing antibodies.

In some embodiments, non-limiting exemplary cell surface markers include CD298, 32 microglobulin, CD45, and MHC class I molecules.

The invention further contemplates systems configured to carry out any of the methods described herein, as well as compositions obtained from the methods and/or systems.

### Systems of the Invention

In one aspect, the invention provides a system for processing multiple cells that enables analysis of DNA and mRNA originating from an individual cell in a biological sample, the system comprising: a) a transposome-nucleic acid assembly comprising a transposase and a nucleic acid molecule comprising an adapter sequence located at a single stranded 3′ end on a non-transfer strand of the nucleic acid molecule, wherein said transposome-nucleic acid complex is configured to generate a plurality of adapter-flanked genomic DNA fragments having the adapter sequence located at a single stranded 3′ overhang end of the non-transfer strand of the plurality of adapter-flanked genomic DNA fragments in a plurality of cells, the adapter sequence comprising a sequence complementary to a capture sequence of a third oligonucleotide, the third oligonucleotide comprising one or more of a barcode sequence, a cell barcode sequence, and/or a unique molecular identifier (UMI); b) a reverse transcriptase component configured to generate cDNA from mRNA; c) a gap-filling component configured to fill gaps in the plurality of adapter-flanked genomic DNA fragments; d) a ligation component configured to seal nicks in the plurality of adapter-flanked genomic DNA fragments; and e) a microfluidic device comprising a receiving component that receives the plurality of cells, wherein a cell of the plurality of cells comprises at least one adapter-flanked genomic DNA fragment of the plurality of adapter-flanked genomic DNA fragments, wherein each of the plurality of adapter-flanked genomic DNA fragments is indicative of a region of accessible chromatin within the cell, and a channel structure that partitions (i) the plurality of cells; and (ii) a plurality of barcoded beads into a plurality of droplets, wherein the plurality of barcoded beads comprises a plurality of nucleic acid molecules comprising barcode sequences, and the third oligonucleotide, wherein a droplet of the plurality of droplets comprises: (1) a cell of the plurality of cells comprising at least one adapter-genomic DNA fragment and/or at least one cDNA, and (2) a barcoded bead of the plurality of barcoded beads, wherein the barcoded bead comprises nucleic acid molecules comprising a common barcode sequence, wherein the common barcode sequence of the nucleic acid molecules of the barcoded bead differs from nucleic acid barcode sequences of other barcoded beads in other droplets of the plurality of droplets; and wherein the system is capable of generating a barcoded, adapter-flanked nucleic acid fragment comprising the common barcode sequence, and/or a barcoded cDNA fragment comprising the common barcode sequence.

In some embodiments, the transposome-nucleic acid assembly comprises: the transposase; a first oligonucleotide comprising a free 5′ phosphate at a 5′ end of the first oligonucleotide and a transposase recognition site; and a second oligonucleotide comprising a free 5′ phosphate at a 5′ end of the second oligonucleotide, the transposase recognition site, and a sequence complementary to the capture sequence of the third oligonucleotide at a 3′ end of the second oligonucleotide, wherein the transposome-nucleic acid assembly is further configured to allow annealing of the first oligonucleotide and second oligonucleotide to one another to form a plurality of adaptors that contain a double-stranded transposase recognition site and a single-stranded 3′ overhang on a non-transfer strand, the single-stranded 3′ overhang comprising the sequence complementary to the capture sequence of the third oligonucleotide.

In some embodiments, the second oligonucleotide comprises a modification at the 3′ end to prevent degradation of the oligonucleotide, optionally wherein the modification is one or more phosphorothioate bonds.

In some embodiments, the transposase is a Tn5 transposase or a hyperactive Tn5 transposase or a functional mutant or derivative of any of said transposases, or a MuA transposase or a functional mutant or derivative thereof, and wherein the transposase recognition site is a Tn5 recognition site or a MuA recognition site comprising a R1 sequence and a R2 sequence.

In some embodiments, the transposase is a Tn5 transposase or a hyperactive Tn5 transposase or a functional mutant or derivative of any of said transposases, and wherein the transposase recognition site is a Tn5 recognition site.

In some embodiments, the system further comprises a labeling component configured to label the cell with oligonucleotide-labeled lipids, oligonucleotide-tagged hashing antibodies, or oligonucleotide-tagged antibodies or against specific surface markers. In some embodiments, non-limiting exemplary cell surface markers include CD298, 32 microglobulin, CD45, and MHC class I molecules.

In some embodiments, the cell is labeled with oligonucleotide-tagged antibodies against specific surface markers by a method comprising: generating antibody-derived tag (ADT) fragments from the oligonucleotide-tagged antibodies or the oligonucleotide-tagged hashing antibodies, the ADT fragments being captured by the third oligonucleotide attached to the carrier; purifying the ADT fragments; amplifying the ADT fragments and separating the amplified ADT fragments from the amplified genomic DNA fragments and/or cDNA; and preparing an ADT library from the amplified ADT fragments.

In some embodiments, the system further comprises a RNase inhibitor.

In some embodiments, the transposase is inactivated after generating the plurality of adapter-flanked genomic DNA fragments.

In some embodiments, the reverse transcriptase enzyme is inactivated after generating the plurality of cDNA fragments and ADT fragments.

In some embodiments, the transposase is inactivated by heating to 65° C., incubating with 0.04% SDS at room temperature, or by the addition of EDTA.

In some embodiments, the gap filling polymerase component is a non-hot start non-strand-displacing DNA polymerase.

In some embodiments, the ligation component is T4 DNA ligase or E. coli DNA ligase.

In some embodiments, the nucleic acid molecules comprising the common barcode sequence further comprise a sequence complementary to the adapter sequence and/or the tag.

In some embodiments, the cDNA is used to amplify a V(D)J region of transcripts using specific primers.

In some embodiments, the ligation component is capable of ligating a nucleic acid molecule comprising the common barcode sequence to each of the plurality of adapter-flanked genomic DNA fragments to generate a barcoded, adapter-flanked genomic DNA fragment.

In some embodiments, the system further comprises an amplification component comprising a polymerase enzyme, wherein the amplification component is configured to use a nucleic acid molecule comprising the common barcode sequence and each of the plurality of adapter-flanked genomic DNA fragments, ADT fragments, V(D)J fragments, and cDNA to generate a barcoded, adapter-flanked genomic DNA fragment, a barcoded ADT fragment, a barcoded V(D)J fragment, and/or a barcoded cDNA fragment.

In some embodiments, the adapter sequence comprises a first sequencing primer sequence, wherein the tag comprises a second sequencing primer sequence, and wherein the first sequencing primer sequence is a different sequence than the second sequencing primer sequence.

In some embodiments, the system further comprises a sequencing component comprising a sequencing instrument capable of generating a plurality of sequencing reads corresponding to the adapter-flanked nucleic acid fragment, the ADT fragment, the V(D)J fragment and/or the cDNA fragment.

In some embodiments, the system is further configured to analyze surface protein information, such as surface epitopes and/or immune receptors on the cell.

In some embodiments, the system further comprises a computing component comprising a processor, a user interface, and an electronic display, wherein the computing component is configured to analyze the sequencing reads and surface protein information and display an analysis of nucleic acid sequencing data and surface protein data on the electronic display.

In some embodiments, the analysis comprises a representation of accessible chromatin, the transcriptome profile, the surface epitopes, and/or the immune receptor repertoire of the cell.

In another aspect, the invention provides a system for processing multiple cells that enables analysis of DNA, surface markers, and mRNA originating from an individual cell, comprising: a) a transposome-nucleic acid assembly comprising a transposase and a nucleic acid molecule comprising an adapter sequence located at a single stranded 3′ end on a non-transfer strand of the nucleic acid molecule, wherein said transposome-nucleic acid complex is configured to generate a plurality of adapter-flanked genomic DNA fragments having the adapter sequence located at a single stranded 3′ overhang end of the non-transfer strand of the plurality of adapter-flanked genomic DNA fragments in a plurality of cells, the adapter sequence comprising a sequence complementary to a capture sequence of a third oligonucleotide, the third oligonucleotide comprising one or more of a barcode sequence, a cell barcode sequence, and/or a unique molecular identifier (UMI); b) an oligonucleotide-tagged lipid or oligonucleotide-tagged antibody tagging component configured to label cells with the oligonucleotide-tagged lipids, oligonucleotide-tagged hashing antibodies, or oligonucleotide-tagged antibodies against specific surface markers; c) a reverse transcriptase component configured to generate cDNA from mRNA and to generate antibody derived tag (ADT) fragments from the oligonucleotide-tagged hashing antibodies or the oligonucleotide-tagged antibodies; d) a gap-filling component configured to fill gaps in the plurality of adapter-flanked genomic DNA fragments; e) a ligation component configured to seal nicks in the plurality of adapter-flanked genomic DNA fragments; and f) a microfluidic device comprising a receiving component that receives the plurality of cells, wherein a cell of the plurality of cells comprises at least one adapter-flanked genomic DNA fragment of the plurality of adapter-flanked genomic DNA fragments, wherein each of the plurality of adapter-flanked genomic DNA fragments is indicative of a region of accessible chromatin within the cell, and a channel structure that partitions (i) the plurality of cells; and (ii) a plurality of barcoded beads into a plurality of droplets, wherein the plurality of barcoded beads comprises a plurality of nucleic acid molecules comprising barcode sequences, and the third oligonucleotide, wherein a droplet of the plurality of droplets comprises: (1) a cell of the plurality of cells comprising at least one adapter-flanked genomic DNA fragment, at least one ADT fragment, and/or at least one cDNA, and (2) a barcoded bead of the plurality of barcoded beads, wherein the barcoded bead comprises nucleic acid molecules comprising a common barcode sequence, wherein the common barcode sequence of the nucleic acid molecules and the ADT fragments of the barcoded bead differs from nucleic acid barcode sequences of other barcoded beads in other droplets of the plurality of droplets; and wherein the system is capable of generating a barcoded, adapter-flanked genomic DNA fragment comprising the common barcode sequence, a barcoded ADT fragment comprising the common barcode sequence, and/or a barcoded cDNA comprising the common barcode sequence.

In some embodiments, the transposome-nucleic acid assembly comprises: the transposase; a first oligonucleotide comprising a free 5′ phosphate at a 5′ end of the first oligonucleotide and a transposase recognition site; and a second oligonucleotide comprising a free 5′ phosphate at a 5′ end of the second oligonucleotide, the transposase recognition site, and a sequence complementary to the capture sequence of the third oligonucleotide at a 3′ end of the second oligonucleotide, wherein the transposome-nucleic acid assembly is further configured to allow annealing of the first oligonucleotide and second oligonucleotide to one another to form a plurality of adaptors that contain a double-stranded transposase recognition site and a single-stranded 3′ overhang on a non-transfer strand, the single-stranded 3′ overhang comprising the sequence complementary to the capture sequence of the third oligonucleotide.

In some embodiments, the second oligonucleotide comprises a modification at the 3′ end to prevent degradation of the oligonucleotide, optionally wherein the modification is one or more phosphorothioate bonds.

In some embodiments, the transposase is a Tn5 transposase or a hyperactive Tn5 transposase or a functional mutant or derivative of any of said transposases, or a MuA transposase or a functional mutant or derivative thereof, and wherein the transposase recognition site is a Tn5 recognition site or a MuA recognition site comprising a R1 sequence and a R2 sequence.

In some embodiments, the transposase is a Tn5 transposase or a hyperactive Tn5 transposase or a functional mutant or derivative of any of said transposases, and wherein the transposase recognition site is a Tn5 recognition site.

In some embodiments, the system further comprises a RNase inhibitor.

In some embodiments, the transposase is inactivated after generating the plurality of adapter-flanked genomic DNA fragments.

In some embodiments, the reverse transcriptase enzyme is inactivated after generating the plurality of cDNA fragments and ADT fragments.

In some embodiments, the transposase is inactivated by heating to 65° C., incubating with 0.04% SDS at room temperature, or by the addition of EDTA.

In some embodiments, the gap filling polymerase component is a non-hot start non-strand-displacing DNA polymerase.

In some embodiments, the ligation component is T4 DNA ligase or E. coli DNA ligase.

In some embodiments, the nucleic acid molecules comprising the common barcode sequence further comprise a sequence complementary to the adapter sequence and/or the tag.

In some embodiments, the cDNA fragments are used to amplify a V(D)J region of transcripts using specific primers.

In some embodiments, the ligation component is capable of ligating a nucleic acid molecule comprising the common barcode sequence to each of the plurality of adapter-flanked genomic DNA fragments to generate a barcoded, adapter-flanked genomic DNA fragment.

In some embodiments, the system further comprises an amplification component comprising a polymerase enzyme, wherein the amplification component is configured to use a nucleic acid molecule comprising the common barcode sequence and each of the plurality of adapter-flanked genomic DNA fragments, ADT fragments, V(D)J fragments, and cDNA to generate a barcoded, adapter-flanked genomic DNA fragment, a barcoded ADT fragment, a barcoded V(D)J fragment, and/or a barcoded cDNA.

In some embodiments, the adapter sequence comprises a first sequencing primer sequence, wherein the tag comprises a second sequencing primer sequence, and wherein the first sequencing primer sequence is a different sequence than the second sequencing primer sequence.

In some embodiments, the system further comprises a sequencing component comprising a sequencing instrument capable of generating a plurality of sequencing reads corresponding to the adapter-flanked nucleic acid fragment, the ADT fragment, the V(D)J fragment and/or the cDNA fragment.

In some embodiments, the system is further configured to analyze surface protein information, such as surface epitopes and/or immune receptors on the cell.

In some embodiments, the system further comprises a computing component comprising a processor, a user interface, and an electronic display, wherein the computing component is configured to analyze the sequencing reads and surface protein information and display an analysis of nucleic acid sequencing data and surface protein data on the electronic display.

In some embodiments, the analysis comprises a representation of accessible chromatin, the transcriptome profile, the surface epitopes, and/or the immune receptor repertoire of the cell.

In some embodiments, non-limiting exemplary cell surface markers include CD298, 32 microglobulin, CD45, and MHC class I molecules.

### Compositions of the Invention

In another aspect, the invention relates to a composition comprising: a transposase; a first oligonucleotide comprising a free 5′ phosphate at a 5′ end of the first oligonucleotide and transposase recognition site; and a second oligonucleotide comprising a free 5′ phosphate at a 5′ end of the second oligonucleotide, the transposase recognition site, and a sequence complementary to the capture sequence of the third oligonucleotide at a 3′ end of the second oligonucleotide on the non-transfer strand, wherein the transposome-nucleic acid assembly is further configured to allow annealing of the first oligonucleotide and second oligonucleotide to one another to form a plurality of adaptors that contain a double-stranded transposase recognition site and a single-stranded 3′ overhang on the non-transfer strand, the single-stranded 3′ overhang comprising the sequence complementary to the capture sequence of the third oligonucleotide, and wherein the composition is configured to produce a plurality of tagmented genomic DNA fragments with a transposase recognition site and a complementary sequence to a capture sequence of a third oligonucleotide, the complementary sequence located at a single stranded 3′ end on a non-transfer strand of the tagmented genomic DNA, wherein the third oligonucleotide is attached to a bead and further comprises at least one of a barcode sequence, a cell barcode sequence, and a unique molecular identifier (UMI).

In some embodiments, the second oligonucleotide comprises a modification at the 3′ end to prevent degradation of the oligonucleotide, optionally wherein the modification is one or more phosphorothioate bonds.

In some embodiments, the transposase is a Tn5 transposase or a hyperactive Tn5 transposase or a functional mutant or derivative of any of said transposases, or a MuA transposase or a functional mutant or derivative thereof, and wherein the transposase recognition site is a Tn5 recognition site or a MuA recognition site comprising a R1 sequence and a R2 sequence.

In some embodiments, the transposase is a Tn5 transposase or a hyperactive Tn5 transposase or a functional mutant or derivative of any of said transposases, and wherein the transposase recognition site is a Tn5 recognition site.

In another aspect, the invention relates to a library of tagmented genomic DNA fragments produced by any of the methods of the present disclosure.

### EXAMPLES

The present invention is also described and demonstrated by way of the following examples. However, the use of these and other examples anywhere in the specification is illustrative only and in no way limits the scope and meaning of the invention or of any exemplified term. Likewise, the invention is not limited to any particular preferred embodiments described here. Indeed, many modifications and variations of the invention may be apparent to those skilled in the art upon reading this specification, and such variations can be made without departing from the invention in spirit or in scope. The invention is therefore to be limited only by the terms of the appended claims along with the full scope of equivalents to which those claims are entitled.

### Example 1. SCRATCH-Seq (Single-Cell Immune Repertoire, Antigen, Transcription, Chromatic Accessibility by Sequencing)

Tumor development and progression are influenced by genetic and epigenetic changes within the tumor cells and dynamic interaction of cancer cells with the tumor microenvironment (TME) (1-5). Immune cells are an important component of the TME. Therefore, reciprocal signaling between malignant cells and immune cells in the TME can alter antitumor immune responses and regulate disease progression.

Antigen-specific B and T lymphocytes play an important role in antitumor immune responses (1,4). The antigen receptors determine the specificity of the tumor-infiltrating B and T lymphocytes. The diversity of B cell and T cell receptors (BCR and TCR, respectively) is achieved through rearrangement of the variable (V), diversity (D), and joining (J) genes through a process called V(D)J recombination (6). Antigen receptor repertoire analysis is important for assessment of clonality of malignant cells in lymphomas, as well as for evaluation of antigen-specific anti-tumor responses in both hematological malignancies and solid tumors (7). TCR and BCR repertoire profiling of individual cells can be used to measure lymphocyte dynamics following interventions such as cancer immunotherapy to evaluate clinical response to treatment and disease prognosis (8).

Epigenetic regulation of chromatin accessibility is needed for the normal development and maintenance of cell-specific gene expression. Disruption of these mechanisms can lead to altered gene expression and malignant transformation. Epigenetic changes are found in all human cancers and mutations are frequently observed in genes that modify the epigenome (2,3). Global changes in the epigenetic landscape can play a significant role in cancer development and progression (3,5,9), and epigenetic therapies are emerging as a promising therapeutic approach for treatment of hematological malignancies and are also being evaluated in the context of solid tumors (10,11).

Epigenetic alterations and subsequent transcriptional changes contribute to immune evasion by the malignant cells (12). The ability to simultaneously assess chromatin accessibility and V(D)J rearrangements in individual cells can provide insights into the complex interactions between tumor cells and immune cells in the TME. Improved understanding of molecular mechanisms of immune evasion and resistance to current therapies can lead to improved treatment of malignancies.

Massively parallel single cell methods have advanced from single modal measurements of transcriptomes alone to bimodal platforms capable of simultaneously measuring transcriptome and surface epitopes (CITE-seq, REAP-seq (13)), transcriptome and chromatin accessibility (SNARE-seq (14), SHARE-seq (15)), and chromatin accessibility and surface epitopes (ASAP-seq (16), ICICLE-seq (17)) and trimodal planforms that allow simultaneous measurement of transcriptome, surface epitopes, and chromatin accessibility (DOGMA-seq (16), TEA-seq (17)). Adding the layer of chromatin accessibility to expanded Cellular Indexing of Transcriptomic and Epitopes by sequencing workflow (exCITE-seq) (18,19) can provide important information that enables more confident assignment of cellular ancestry and sheds light on potential activity and fate of the cell beyond what is afforded by transcriptional information alone.

The methods described herein include the following non-limiting technical innovations:
A method for assessing chromatin accessibility was developed in single cells that is compatible with microfluidic droplet-based technologies that use 5′-based chemistry and allow immune receptor repertoire profiling. The methods described herein allowed unbiased chromatin accessibility and V(D)J profiling of malignant cells and responding immune cells in the TME at single-cell resolution.
A 5′ compatible single-cell ATAC-seq was integrated into the optimized multimodal single-cell pipeline. Incorporation of single-cell ATAC-seq (scATAC-seq) into the exCITE-seq workflow through addition of permeabilization and tagmentation steps may allow characterization of individual cells in the TME.
Analysis and visualization of all parallel modalities was optimized to make the most of the data from the new pipeline that can include transcriptional (GEX), protein/epitope (antibody derived tags—ADT), chromatin accessibility (ATAC-seq), and immune antigen receptor (BCR/TCR and tetramer labeling) information.

Epigenetic changes are present in all human cancers and together with genetic alterations are known to drive tumorigenesis. Such changes occur largely through impairment of the chromatin remodeling machinery leading to accumulation of epigenetic abnormalities. Mutations in genes that modify the epigenome are frequently observed in cancer and lead to altered gene expression of oncogenes and tumor suppressor genes (3). Gene expression programs are partially regulated by sequence-specific interactions between DNA-binding proteins and cis-regulatory elements in the genome. Physical access to DNA is a highly dynamic property of chromatin and plays a critical role in regulation of gene expression (20,21). Chromatin consists of a complex of DNA and histone proteins and has an important role in packaging genomic DNA and regulating DNA functions, such as transcription, replication, recombination, and chromosome segregation (20,22).

The chromatin accessibility landscape of cells is highly dynamic and changes in response to developmental cues and external stimuli (23,24). Nucleosomes are core structural elements of chromatin and consist of a histone octamer wrapped in 147 bp of genomic DNA (22,25). The genomic DNA directly bound to the histone proteins within the nucleosome is usually inaccessible to DNA-binding proteins which function as regulators of transcription. Nucleosome-depleted open chromatin regions define regulatory elements within the genome, and these epigenetic features dynamically regulate gene expression (26). Because nucleosome-bound DNA is protected from cleavage, chromatin accessibility landscape is measured by quantifying the susceptibility of genomic DNA to enzymatic cleavage (20,27).

Assay for transposase-accessible chromatin using sequencing (ATAC-seq) utilizes a hyperactive Tn5 transposase to map nucleosome occupancy (20,27). The Tn5 transposase, loaded with adaptors suitable for downstream sequencing steps, can simultaneously fragment accessible genomic regions and tag fragments with adaptors (tagmentation). Subsequent sequencing of the tagged genomic DNA fragments allows mapping of genomic DNA regions associated with active transcription (20,27). ATAC-seq has been used to interrogate nucleosome positioning in regulatory sites and elucidate networks of cis-regulatory elements (27). Single-cell ATAC-seq has recently been implemented on droplet-based microfluidic platforms (21,28).

Antigen-specific B and T cells play a central role in immune responses to cancer. Repertoire profiling depends on sequencing methods that capture the 5′ end of the transcript to capture the V(D)J sequence of the rearranged variable region genes (29). Information about BCR and TCR rearrangement can further complemented by tetramer labeling that provides information about antigen receptor specificity. Conventional 3′ sequencing approaches produce coverage primarily of the constant region. Because of this, simultaneous chromatin and receptor repertoire profiling of individual cells is not possible with currently existing scATAC-seq methods, which leaves a gap in the ability to characterize normal and malignant immune cells in the TME.

### Results

The exCITE-seq multimodal single-cell platform enables simultaneous capture of surface epitope information and transcriptional profile in individual cells, and since it utilized 5′-based chemistry, it allows for characterization of BCR and TCR rearrangements (18,19). This multi-layered high-throughput approach can be further enhanced by antibody hashing against ubiquitously expressed epitopes (30). Sample hashing enables the combination of multiple samples into one run, reducing batch effects and reagent costs.

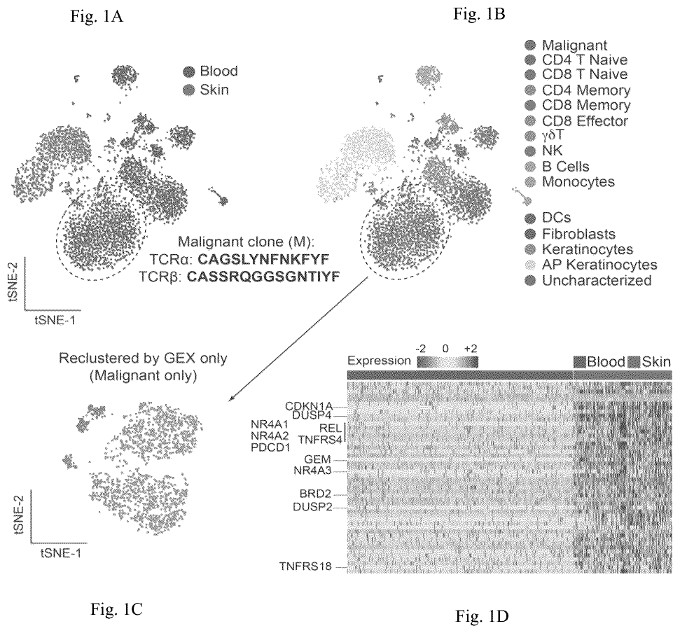

Analysis of blood and skin biopsies from patients with Cutaneous T-cell lymphoma (CTCL) highlighted the tremendous potential of exCITE-seq. The exCITE-seq was used to examine clonal malignant T cells and survey other leukocytes in the blood and skin (FIG. 1). Significant sub-clonal heterogeneity (FIG. 1C) and tissue-specific adaptation was demonstrated by by malignant cells. Furthermore, these findings revealed copy number variation (CNV)-defined sub-clonal populations in matched skin and blood of individual patients, with phylogenetic relationship and transcriptional trajectory analysis revealing tissue-specific adaptation by the malignant cells (31). The exCITE-seq has also been deployed for analysis of TME in lung cancer, where multimodal analysis revealed how tumor mutation in KEAP1 and LKB1 contribute to immune evasion (32).

Develop and Integrate 5′ ATAC-Seq into exCITE-Seq Workflow.
Develop a Single-Cell ATAC-Seq Method Compatible with Microfluidic Droplet-Based Technologies that Capture the 5′ End of the Transcript

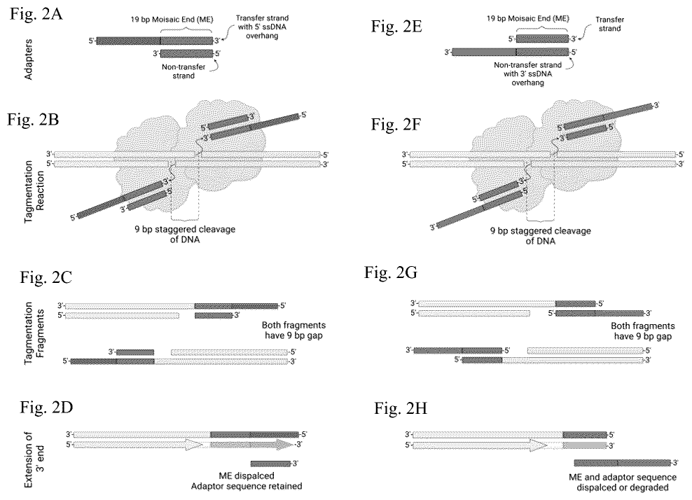

Transposases catalyze the movement of defined DNA elements (transposons) in the genome in a ‘cut and paste’ mechanism (33-35). Tagmentation, in which a hyperactive transposase simultaneously fragments the target DNA and tags the fragments with adaptor sequences, can be used to detect nucleosome-free regions of genomic DNA (36). The single-cell ATAC-seq method utilizes a hyperactive Tn5 transposase to map the distribution of nucleosomes in individual cells (36). Tn5 binds to synthetic 19 bp mosaic end (ME) recognition sequence and tagments genomic DNA, producing DNA fragments that are tagged with the adaptor sequences at both ends (FIG. 2B-2C, 2F-2G). The adaptor itself comprises the ME sequence and an additional single-stranded DNA overhang on one strand (FIG. 2A, 2E). In scATAC-seq methods that use bead-based transcript capture, the single-stranded overhang contains a capture sequence that allows it to bind to the bead oligo and be tagged with a unique molecular identifier (UMI) and cell barcode. All Tn5 tagmentation-based applications use adaptors with a 5′ single-stranded overhang on the transfer strand of the adaptor (i.e., the strand that becomes covalently bound to the genomic DNA (36) (FIG. 2A), however the present data show that the single-stranded overhang can be on either strand (FIG. 2E).

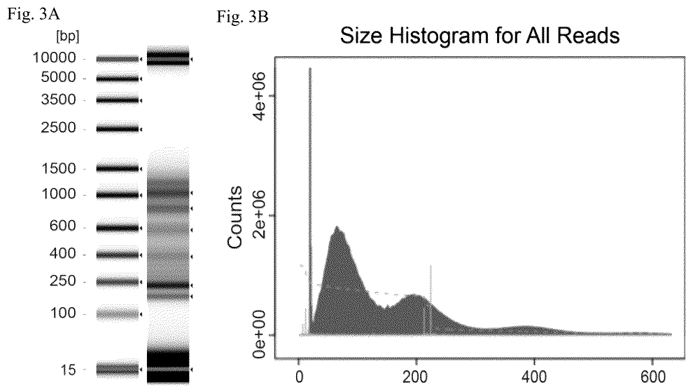

Tagmentation was performed by using Tn5 loaded with 5′ RNA-seq-compatible adaptors in bulk. The genomic DNA fragments produced by tagmentation were amplified using adaptor-specific primers. It was found that the fragment size distribution shows clear periodicity of ˜200 bp (FIG. 3A) which is indicative of nucleosome-bound DNA which is protected from cleavage (27,37). Tagmentation was also performed in single cell emulsions. The ATAC libraries were constructed using Chromium 10× protocol for the Feature barcode library with minor modifications to size selection, and sequenced on Illumina sequencer using standard sequencing primers. Analysis of the insert fragment size revealed a similar distribution with a clear periodicity of ˜200 bp (FIG. 3B). The ATAC-seq analysis was incorporated into the existing pipeline to examine the accessibility of individual loci in different clusters of immune cells. The results demonstrated the feasibility of a scATAC-seq method compatible with 5′-based chemistry.

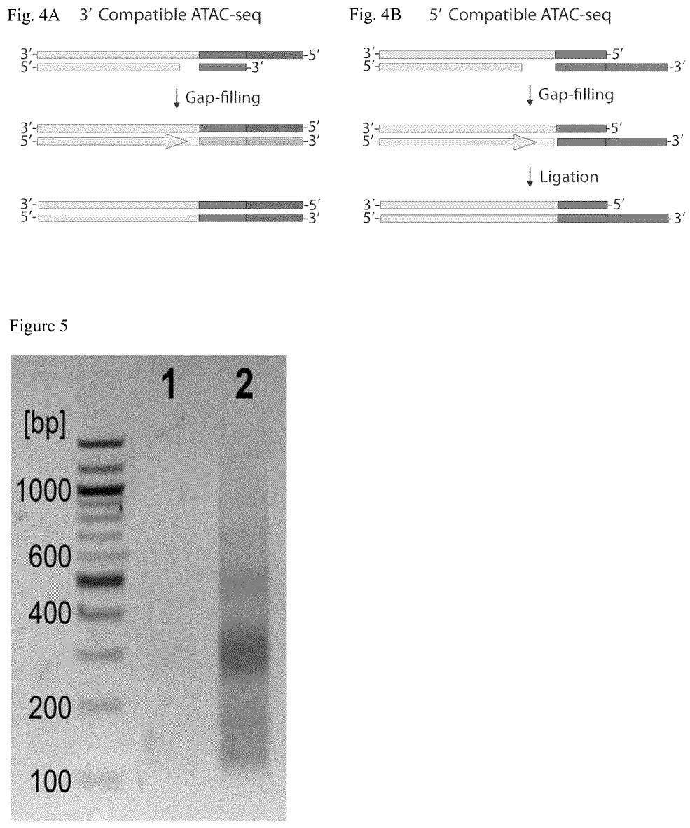

The 9 bp gap left by Tn5 following transposition (38) is a challenge that had to overcome when developing a 5′ scATAC-seq method. In scATAC-seq, the Tn5 adaptors contain sequences complementary to the bead oligo that enable capture and barcoding of the fragment. In methods compatible with 3′ capture (e.g., TEA-seq), gap-filling occurs during the reverse transcription (RT) with no risk of loss of adaptor sequence (FIG. 4A). In 5′ ATAC-seq, the 9 bp gap needs to be filled and the product ligated after reverse transcription to prevent loss of the bead oligo with the UMI and cell barcode (critical for library construction) (FIG. 4B).

The gap-filling reaction was carried out after reverse transcription using a non-hot start non-strand-displacing DNA polymerase (e.g., KAPA HiFi DNA polymerase or T4 DNA polymerase), followed by ligation to seal the nicks in the DNA left after gap-filling (FIG. 3B). A non-limiting exemplary ligation was carried out at room temperature using T7 ligase (NEB) which has high specificity for correctly base-paired nicks if used with T4 DNA ligase buffer (NEB) and supplemented with ATP. Another non-exemplary ligation was carried out at 16° C. using room temperature using E. coli DNA ligase. Following ligation, the ATAC fragments were amplified normally using primers specific to the adaptor sequence.

### Workflow

### I. Transposome Assembly

a. Oligos were designed and annealed to make adaptors that contain a double-stranded mosaic end (ME) sequence and a single-stranded 3′ overhang with sequence complementary to the capture sequence of the bead oligo of Chromium Next GEM Single Cell 5′ Gel Beads v2, a free phosphate at the 5′ end, and two phosphorothioate bonds at the 3′ end to protect the oligo from nuclease degradation (Table 1). Lyophilized oligos were purchased from Integrated DNA Technologies, Inc.

TABLE 1

| Description | Sequence |
|---|---|
| ME with 5′ phos- | 5′-/5Phos/CTGTCTCTTATACACATCT-3′ |
| phate | (SEQ ID NO: 1) |
| ME with 5′ phos- | 5′-/5Phos/CTGTCTCTTATACACATCTCC |
| phate and 3′ | CATATAAGA*A*A-3′ (SEQ ID NO: 2) |
| capture sequence | |
| complementary | |
| overhang | |

b. The oligos were resuspended in 40 mM Tris-HCl (pH8.0), 50 mM NaCl buffer at 100 mM concentration. The oligos were annealed in a thermal cycler to generate the adaptors (Table 2). In a PCR tube, the two oligos were mixed 1:1, heated to 95° C., cooled to 65° C. by lowering the temperature −0.1° C./second, held at 65° C. for 5 minutes, and finally cooled to 4° C. by lowering the temperature −0.1° C./second.

TABLE 2
3′-GACAGAGAATATGTGTAGA/Phos/-5′ (SEQ ID NO: 4)
5′-/5Phos/CTGTCTCTTATACACATCTCCCATATAAGA*A*A-3′
(SEQ ID NO: 2)

c. The Tn5 was loaded with the custom adaptors in a thermal cycler. Unloaded Tn5 (Diagenode) was mixed with annealed oligos (1 mg Tn5 with 1 ml adaptor) and incubated at 23° C. for 30 minutes.

### II. Permeabilize Cells and Make Emulsion

a. Live or fixed cells were permeabilized with 20 mM Tris-HCl pH7.4, 150 mM NaCl, 3 mM MgCl2, and 0.01%-0.015% digitonin for 5 minutes on ice then washed in 20 mM Tris-HCl pH7.4, 150 mM NaCl, 3 mM MgCl2 buffer.
b. After permeabilization, cells were incubated at 37° C. for 60 minutes with loaded Tn5 in 1× tagmentation buffer (Diagenode) and 1 U/ml RNase inhibitor.
c. After tagmentation, the cell suspension was incubated for 5 minutes at room temperature to remove the Tn5 enzyme from DNA. The cell suspension was diluted so the final SDS concentration is less than 0.005% to avoid inhibiting the reverse transcriptase (RT) enzyme.
d. After incubation, cells were counted, resuspended at a concentration of 150-1,500 cells/μl, and processed for single-cell RNA-seq using the Chromium Next GEM Single Cell 5′ kit v2 according to the manufacturer's protocol. The reverse transcription reaction was carried out according to the 10× Genomics protocol. The emulsion was incubated in a thermal cycler at 53° C. for 45 minutes, then 85° C. for 5 minutes.
e. The cDNA and genomic DNA fragments were cleaned up according to 10× protocol.

### III. Gap-Filling and Ligation

a. A non-limiting exemplary gap-filling was done by incubating the cDNA/genomic DNA fragments with KAPA HIFI non-hot start polymerase at 37° C. for 10 minutes. Another non-limiting exemplary gap-filling was done by incubating the cDNA/genomic DNA fragments with T4 DNA polymerase at 12° C. for 15 minutes. Fragments were cleaned up using SPRISelect reagent or MyOne SILANE Dynabeads according to the manufacturer's instructions and eluted in EB buffer.
b. In a nonlimiting example, the eluted DNA was incubated with T7 DNA ligase in 1×T4 DNA ligase buffer supplemented with 1 mM ATP for 15 minutes at room temperature. In another non-limiting example, the eluted DNA was incubated with E. coli DNA ligase 16° C. for 30 minutes then 65° C. for 20 minutes. Fragments were cleaned up using SPRISelect reagent or MyOne SILANE Dynabeads according to the manufacturer's instructions and eluted in EB buffer.
c. Alternatively, gap-filling and nick-sealing can be combined in one step by incubating genomic DNA fragments with T4 DNA polymerase and Ampligase at 30° C. for 30 minutes or E. coli ligase at 16° C. for 30 minutes. Fragments are cleaned with SPRISelect reagent and eluted in EB buffer.
d. Unloaded Tn5 transposase and tagmentation buffer were purchased from Diagenode.

### IV. Library Preparation

a. cDNA and ATAC fragments were amplified with the appropriate primers.
b. After size selection, ATAC library was constructed using indexing primers containing Truseq Read 1 and Nextera Read 2 sequences for sequencing on Illumina sequencers using standard primers.
   Integrate 5′ scATAC-Seq into Multimodal exCITE-Seq Workflow without Compromising the Other Modalities

Joint analysis of multimodal data facilitated the ability to detect cis- and trans-regulatory elements underlying the identity and transcriptional trajectory of individual cells. To that end, the inventors' goal was to integrate 5′ scATAC-seq into a multimodal exCITE-seq workflow.

Successful integration of 5′ scATAC-seq into the multimodal exCITE-seq platform required the inventors to navigate several technical challenges. First, tagmentation requires permeabilization for Tn5 to gain access to the nucleus, yet ensuring that this was done without loss of mRNA diversity and integrity. There are a variety of permeabilization methods used to prepare cells for various downstream applications, including scATAC-seq (17). Permeabilization was optimized to allow tagmentation to occur without compromising gene expression profiling and without increasing ambient RNA background, which can affect library quality (39). Concentrations of 0.01% to 0.5% digitonin were found to be suitable for permeabilization, preferably 0.01% to 0.015% digitonin.

The second challenge was optimizing incubation time and concentration of Tn5 for the tagmentation reaction. Because tagmentation fragment length is determined by incubation time and Tn5 concentration (40), the optimal time for the enzymatic reaction and ideal Tn5 concentration for a given cell number was established that produces the desired fragment size with minimal mRNA loss. Tn5 concentrations of 0.03 to 1.3 μg/μl per 50,000 cells was tested with 30 and 60 minute incubation times, and X was found to be the optimal concentration and X was found to be the optimal incubation time.

Another challenge was the ability to separate tagmentation fragments from cDNA and ADTs to make the corresponding libraries. ADTs were separated from cDNA fragments through size selection using the Solid Phase Reversible Immobilization (SPRI) method (41). This was possible because ADTs are <200 bp while cDNA fragments of interest are >400 bp. However, tagmentation fragments can range from 50-800 bp. Because these sizes overlap with cDNA and ADTs, tagmentation fragments were separated from cDNA and ADTs before size selection. Amplifying tagmentation fragments with an oligonucleotide conjugated to d-Desthiobiotin allowed the inventors to pull down those fragments from the mixture using streptavidin bound to magnetic beads. The bound fragments were then eluted from the beads in water by incubating at 95° C. for 15 minutes and. free fragments which were recovered from the supernatant. Amplification and pull-down steps were optimized and the minimum number of PCR cycles that successfully allows the separation of tagmentation fragments from cDNA and ADTs was determined to minimize PCR-induced sequencing artifacts. Generally, the annealing temperature depends on the sequence of the specific primer used. Four to six PCR cycles appeared to be sufficient to capture most fragments, but the number can be varied depending on the specific tagmentation. An ATAC-seq library was amplified with biotinylated primer and the biotinylated products were captured using commercially available streptavidin magnetic beads (FIG. 5), demonstrating the feasibility of this method.

Finally, the integration of the multimodal single-cell data was optimized. The current analysis pipeline has been described in a few recent publications (18,19,31). Briefly, the Cell Ranger software suite from 10× Genomics was used to demultiplex cellular barcodes, align reads to the human genome (GRCh38), and perform UMI counting for GEX and V(D)J libraries. ADT count matrices was generated by kallisto kb-count v0.24.1 (42,43) and samples demultiplexed using HTODemux( ) from the R package Seurat (44). To process the 5′ scATAC-seq data, an approach based on pseudo alignment using kallisto and quantified with bustools as previously described (45) was utilized. Data integration, visualization, and analysis were done using the Seurat toolkit (44,46).

The experiments described herein have demonstrated the feasibility of performing tagmentation using a Tn5 transposase loaded with adaptors carrying a 3′ ssDNA overhang in bulk and in single cells using a microfluidic bead-based chemistry. Using the described method, the inventors have successfully constructed and sequenced scATAC-seq libraries which display the expected fragment size distribution characteristic of successful tagmentation of native chromatin.

Integration of scATAC-seq into the multimodal exCITE-seq platform also depends on successful capture and adequate separation of ATAC and ADT fragments during library preparation. Because the construction of ADT and ATAC libraries uses the same flanking sequences, it is possible that some smaller (<300 bp) ATAC fragments can contaminate the ADT library. The ADT library contains tags from antibodies used to detect various surface antigens and those used to hash the samples before multiplexing. Contaminating fragments from the tagmentation reaction can be filtered out in pre-processing because they do not contain a valid antibody barcode and thus may not impair the ability to demultiplex samples during analysis. The ADT libraries may require deeper sequencing to ensure there are sufficient reads to perform demultiplexing and surface epitope analysis, however that is a small fraction of total sequencing depth and cost.

### Validation and Benchmarking of the Integrated Multimodal Single-Cell Platform.

A comprehensive evaluation of the present new integrated multimodal platform was performed. In addition, the integrated platform was benchmarked against trimodal TEA-seq assay (17) and current exCITE-seq platform using PBMCs collected from heathy donors before and after in vitro stimulation.

The PBMCs were stimulated in vitro, then stained and processed according to the protocol for each assay using the appropriate oligonucleotide-tagged antibodies and 10× Chromium reagents. Reverse transcription reaction, fragment amplification, V(D)J enrichment, and construction of all libraries were proceeded according to the previously established protocols (18,19,31). The libraries were quantified, pooled, and sequenced on an Illumina sequencer.

The data analysis and benchmarking were done as described by Xu et.al. (49). Data preprocessing for integrated 5′ ATAC-seq and exCITE-seq were done as described above. All ADT count matrices were generated by kallisto kb-count v0.24.1 (42,43) and sample demultiplexing was performed using HTODemux (46). 3′ ATAC libraries for TEA-seq were processed together with GEX libraries using the Cell Ranger ARC software suite. 5′ ATAC libraries were processed as described (45). Finally, key performance metrics from the Cell Ranger and kallisto outputs were used to compare the different methods as outlined below.

Library complexity analysis estimates the number of unique molecules for a given number of total molecules observed for the cell, according to the Lander-Waterman equation (50), with low numbers suggestive of inadequate amounts of starting material (cDNA, genomic DNA fragments, etc.) or losses during cleanup or size selection. Reduction in library complexity compromises downstream analysis. Complexity was calculated for each library to assess the performance of each modality between the different assays.

Ambient RNA contamination occurs when ambient RNA gets incorporated into droplets during Gel bead-in Emulsion (GEM) generation and is barcoded and amplified along with the cell's native mRNA. This results in cross-contamination of transcripts between different cell populations and can confound downstream analysis. DecontX (39) was used to estimate ambient RNA contamination for each cell under the different processing conditions. Ambient RNA contamination is especially a concern for protocols that involve cell permeabilization which can allow leakage of mRNA from the cell.

To evaluate the surface epitope detection rate, the fraction of cells with UMIs>0 was calculated for all protein tags in the cocktail. Pairwise comparison of ADT detection frequencies and ADT fold change between the different assays was performed to determine if there was impaired ADT detection as a result of permeabilization and whether it attenuates differential ADT signal after stimulation.

The different methods were assessed by cluster analysis. For all methods, clustering wasw performed using RNA, ADT, and ATAC data separately. Unsupervised ‘weighted-nearest neighbor’ (WNN) analysis was performed to leverage all three types of data using Seurat (46). Finally, purity assessment for all identified cell clusters was performed using ROGUE, an entropy-based statistic, to determine cluster cell purity and compare the different methods (51).

Chromatin states are functionally defined by histone modifications and transcription factor binding in addition to DNA accessibility. Most current scATAC-seq methods are limited to only measuring accessible chromatin. Further development of the platform can incorporate nanobody-tethered tagmentation which utilizes nanobody-Tn5 fusion proteins that recognize specific histone post-translational modifications (52) allowing a more comprehensive profiling of chromatin states and protein-DNA binding sites.

### Performance Measures:

### ATAC-Seq Libraries:

Nucleosome banding pattern: the histogram of DNA fragment sizes should exhibit a strong nucleosome banding pattern. Lack of banding pattern indicates loss of native chromatin structure.
Fraction of reads mapped to mitochondrial genome<20%: Higher number could indicate a problem with cell permeabilization leading to disruption of mitochondrial membrane.
Fragments in nucleosome-free regions (fragments smaller than 124 bp)>40%: Low percentage could indicate incorrect ratio of Tn5 to cell number leading to insufficient fragmentation and fragments that are larger than expected. It could also indicate problems with size selection during library preparation.
Fragments flanking multiple nucleosomes (fragment size>400 bp)<5%: High percent of reads mapping to multiple nucleosomes could indicate incorrect ratio of Tn5 to cell number leading to incomplete fragmentation. It could also indicate problems with size selection during library preparation.

### GEX/V(D)J Libraries:

Fraction of reads with valid barcodes correctly mapped to transcriptome>70%: Lower percentage indicates a high level of ambient RNA.
Median genes per cell>1000: Low gene per cell yield indicates poor quality sample or inefficient transcript capture. Precise value is cell type dependent.
Reads mapped to V(D)J>18%: Determined experimentally for PBMCs. Lower than expected values indicate a problem with transcript capture or V(D)J enrichment.
   Benchmarking Against Current exCITE-Seq and TEA-Seq:
Library complexity: Difference in log₁₀ UMI complexity for each library 15% between integrated 5′ ATAC-seq and TEA-seq and 25% between integrated 5′ ATAC-seq and exCITE-seq.
Ambient RNA contamination<25%: The median ambient RNA contamination in singlets in a published PBMC single-cell dataset was 7.02% (0.07-65%) according to Yang et. al.³⁹. Permeabilization is expected to lead to higher levels of ambient RNA due to mRNA leakage from permeabilized cells
Surface epitope detection: Protein tag detection frequencies for integrated 5′ ATAC-seq within 10% of those for TEA-Seq. Fold change (log₂FC) observed for activation-induced markers for integrated 5′ ATAC-seq within 10% of those for TEA-Seq.
Average ROGUE (cluster purity) for integrated 5′ ATAC-seq>0.6 for ADT, >0.55 for RNA, and >0.6 for trimodal WNN clustering. All values should be within 20% of ROGUE for TEA-seq for each library and within 30% for exCITE-seq.

### Example 2. Alternative Method for Tagmentation and Separating ATAC Fragments from cDNA

One of the oligonucleotides used to make the transposase assembly (e.g., an assembly comprising Tn5, hyperactive Tn5, and functional mutants and derivatives thereof) adaptors contains an internal nucleotide conjugated to d-Desthiobiotin. This way the tagmented genomic DNA fragments produced will already be tagged with biotin. In this method, after gap filling and nick-sealing, biotinylated fragments can be pulled down directly without the need for an additional amplification step with biotinylated primer.

### Workflow

### I. Transposome Assembly

a. Oligonucleotides are designed and annealed to make adaptors that contain a double-stranded mosaic end (ME) sequence and a single-stranded 3′ overhang with sequence complementary to the capture sequence of the bead oligonucleotides of Chromium Next GEM Single Cell 5′ Gel Beads v2, an internal d-Desthiobiotin modification in the region complementary to the capture sequence, a free phosphate at the 5′ end, and at least three phosphorothioate bonds at the 3′ end to protect the oligo from nuclease degradation (Table 1). Lyophilized oligonucleotides are purchased from Integrated DNA Technologies, Inc.

TABLE 1

| Description | Sequence |
|---|---|
| ME with 5′ phos- | 5′-/5Phos/CTGTCTCTTATACACATCT-3′ |
| phate | (SEQ ID NO: 1) |
| ME with 5′ phos- | 5′-/5Phos/CTGTCTCTTATACACATCTCCC |
| phate and 3′ | ATATAA/ideSBioTEG/G*A*A*A-3′ |
| capture sequence | (SEQ ID NO: 3) |

°indicates internal d-Desthiobiotin modification
*indicates phosphorothioate bond

b. The oligonucleotides are resuspended in 40 mM Tris-HCl (pH8.0), 50 mM NaCl buffer at 100 μM concentration. The oligonucleotides are annealed in a thermal cycler to generate the adaptors (Table 2). In a PCR tube, the two oligos are mixed 1:1, heated to 95° C., cooled to 65° C. by lowering the temperature −0.1° C./second, held at 65° C. for 5 minutes, and finally cooled to 4° C. by lowering the temperature −0.1° C./second.

TABLE 2
3′-GACAGAGAATATGTGTAGA/Phos/-5' (SEQ ID NO: 4)
5′-/5Phos/CTGTCTCTTATACACATCTCCCATATAA/ideSBioTEG/
G*A*A*A-3′ (SEQ ID NO: 3)

c. The transposase (e.g., Tn5, hyperactive Tn5, and functional mutants and derivatives thereof) is loaded with the custom adaptors in a thermal cycler. Unloaded transposase (e.g., Tn5, hyperactive Tn5, and functional mutants and derivatives thereof) (Diagenode) is mixed with annealed oligonucleotides (2 μl Tn5 with 1 μl adaptor) and incubated at 23° C. for 30 minutes.

### II. Permeabilize Cells and Make Emulsion

a. Live or fixed cells are permeabilized with 20 mM Tris-HCl pH7.4, 150 mM NaCl, 3 mM MgCl2, 0.01%-0.015% digitonin, and RNase inhibitor for 5 minutes on ice then washed in 20 mM Tris-HCl pH7.4, 150 mM NaCl, 3 mM MgCl2 buffer.

b. After permeabilization, cells are incubated at 37° C. for 60 minutes with loaded transposase (e.g., Tn5, hyperactive Tn5, and functional mutants and derivatives thereof) in 1× tagmentation buffer (Diagenode) and 1 U/ml RNase inhibitor.

c. After tagmentation, SDS is added to final concentration of 0.04% and the cell suspension was incubated for 5 minutes at room temperature to remove the transposase enzyme from DNA. The cell suspension is diluted so the final SDS concentration is less than 0.005% to avoid inhibiting the reverse transcriptase (RT) enzyme.

d. After incubation, cells are counted, resuspended at a concentration of 150-1,500 cells/μl, and processed for single-cell RNA-seq using the Chromium Next GEM Single Cell 5′ kit v2 according to the manufacturer's protocol. The reverse transcription reaction is carried out according to the 10× Genomics protocol. The emulsion was incubated in a thermal cycler at 53° C. for 45 minutes, then 85° C. for 5 minutes.

e. The cDNA and genomic DNA fragments are cleaned up according to 10× protocol.

### III. Gap-Filling and Ligation

a. A non-limiting exemplary gap-filling is done by incubating the cDNA/genomic DNA fragments with KAPA HIFI non-hot start polymerase at 37° C. for 10 minutes. Another non-limiting exemplary gap-filling is done by incubating the cDNA/genomic DNA fragments with T4 DNA polymerase at 12° C. for 15 minutes. Fragments are cleaned up using SPRISelect reagent or MyOne SILANE Dynabeads according to the manufacturer's instructions and eluted in EB buffer.

b. In a nonlimiting example, the eluted DNA is incubated with T7 DNA ligase in 1×T4 DNA ligase buffer supplemented with 1 mM ATP for 15 minutes at room temperature. In another non-limiting example, the eluted DNA is incubated with E. coli DNA ligase at 16° C. for 30 minutes then 65° C. for 20 minutes. Fragments are cleaned up using SPRISelect reagent or MyOne SILANE Dynabeads according to the manufacturer's instructions and eluted in EB buffer.

c. Alternatively, gap-filling and nick-sealing can be combined in one step by incubating genomic DNA fragments with T4 DNA polymerase and E. coli ligase at 16° C. for 30 minutes followed by 75° C. for 20 minutes. Fragments are cleaned with SPRISelect reagent and eluted in EB buffer.

d. Unloaded Tn5 transposase and tagmentation buffer are purchased from Diagenode.

### IV. Library Preparation

a. ATAC fragments are separated from cDNA fragments with streptavidin magnetic beads then eluted off the beads in water at 95° C. for 15 minutes, while cDNA fragments are cleaned up from the flowthrough with SPRISelect reagent and eluted in EB buffer.

b. cDNA and ATAC fragments are amplified with the appropriate primers.

c. After size selection, an ATAC library is constructed using indexing primers containing Truseq Read 1 and Nextera Read 2 sequences for sequencing on Illumina sequencers using standard primers.

### REFERENCES

1. Anderson, N. M.and Simon, M. C. The tumor microenvironment. Current Biology 30, R921-R925, doi:https://doi.org/10.1016/j.cub.2020.06.081 (2020).
2. Baxter, E., Windloch, K., Gannon, F. and Lee, J. S. Epigenetic regulation in cancer progression. Cell & Bioscience 4, 45, doi:10.1186/2045-3701-4-45 (2014).
3. Baylin, S. B.and Jones, P. A. Epigenetic Determinants of Cancer. Cold Spring Harb Perspect Biol 8, doi:10.1101/cshperspect.a019505 (2016).
4. Bozyk, A., Wojas-Krawczyk, K., Krawczyk, P. and Milanowski, J. Tumor Microenvironment-A Short Review of Cellular and Interaction Diversity. Biology (Basel) 11, doi:10.3390/biology11060929 (2022).
5. Sharma, S., Kelly, T. K. and Jones, P. A. Epigenetics in cancer. Carcinogenesis 31, 27-36, doi:10.1093/carcin/bgp220 (2010).
6. Roth, D. B. V(D)J Recombination: Mechanism, Errors, and Fidelity. Microbiol Spectr 2, doi:10.1128/microbiolspec.MDNA3-0041-2014 (2014).
7. Gazzola, A., Mannu, C., Rossi, M., Laginestra, M. A., Sapienza, M. R., Fuligni, F., Etebari, M., Melle, F., Sabattini, E., Agostinelli, C., Bacci, F., Sagramoso Sacchetti, C. A., Pileri, S. A. and Piccaluga, P. P. The evolution of clonality testing in the diagnosis and monitoring of hematological malignancies. Ther Adv Hematol 5, 35-47, doi:10.1177/2040620713519729 (2014).
8. Jiang, N., Schonnesen, A. A. and Ma, K. Y. Ushering in Integrated T Cell Repertoire Profiling in Cancer. Trends Cancer 5, 85-94, doi:10.1016/j.trecan.2018.11.005 (2019).
9. Liu, Y., Gonzalez, Y. and Amengual, J. E. Chromatin-Remodeled State in Lymphoma. Curr Hematol Malig Rep 14, 439-450, doi:10.1007/s11899-019-00541-9 (2019).
10. Ansari, J., Shackelford, R. E. and El-Osta, H. Epigenetics in non-small cell lung cancer: from basics to therapeutics. Translational Lung Cancer Research 5, 155-171 (2016).
11. Jin, N., George, T. L., Otterson, G. A., Verschraegen, C., Wen, H., Carbone, D., Herman, J., Bertino, E. M. and He, K. Advances in epigenetic therapeutics with focus on solid tumors. Clinical Epigenetics 13, 83, doi:10.1186/s13148-021-01069-7 (2021).
12. Baghban, R., Roshangar, L., Jahanban-Esfahlan, R., Seidi, K., Ebrahimi-Kalan, A., Jaymand, M., Kolahian, S., Javaheri, T. and Zare, P. Tumor microenvironment complexity and therapeutic implications at a glance. Cell Communication and Signaling 18, 59, doi:10.1186/s12964-020-0530-4 (2020).
13. Peterson, V. M., Zhang, K. X., Kumar, N., Wong, J., Li, L., Wilson, D. C., Moore, R., McClanahan, T. K., Sadekova, S. and Klappenbach, J. A. Multiplexed quantification of proteins and transcripts in single cells. Nature Biotechnology 35, 936-939, doi:10.1038/nbt.3973 (2017).
14. Chen, S., Lake, B. B. and Zhang, K. High-throughput sequencing of the transcriptome and chromatin accessibility in the same cell. Nature Biotechnology 37, 1452-1457, doi:10.1038/s41587-019-0290-0 (2019).
15. Ma, S., Zhang, B., LaFave, L. M., Earl, A. S., Chiang, Z., Hu, Y., Ding, J., Brack, A., Kartha, V. K., Tay, T., Law, T., Lareau, C., Hsu, Y.-C., Regev, A. and Buenrostro, J. D. Chromatin Potential Identified by Shared Single-Cell Profiling of RNA and Chromatin. Cell 183, 1103-1116.e1120, doi:https://doi.org/10.1016/j.cell.2020.09.056 (2020).
16. Mimitou, E. P., Lareau, C. A., Chen, K. Y., Zorzetto-Fernandes, A. L., Hao, Y., Takeshima, Y., Luo, W., Huang, T. S., Yeung, B. Z., Papalexi, E., Thakore, P. I., Kibayashi, T., Wing, J. B., Hata, M., Satija, R., Nazor, K. L., Sakaguchi, S., Ludwig, L. S., Sankaran, V. G., Regev, A. and Smibert, P. Scalable, multimodal profiling of chromatin accessibility, gene expression and protein levels in single cells. Nat Biotechnol 39, 1246-1258, doi:10.1038/s41587-021-00927-2 (2021).
17. Swanson, E., Lord, C., Reading, J., Heubeck, A. T., Genge, P. C., Thomson, Z., Weiss, M. D., Li, X. J., Savage, A. K., Green, R. R., Torgerson, T. R., Bumol, T. F., Graybuck, L. T. and Skene, P. J. Simultaneous trimodal single-cell measurement of transcripts, epitopes, and chromatin accessibility using TEA-seq. eLife 10, doi:10.7554/eLife.63632 (2021).
18. Mimitou, E. P., Cheng, A., Montalbano, A., Hao, S., Stoeckius, M., Legut, M., Roush, T., Herrera, A., Papalexi, E., Ouyang, Z., Satija, R., Sanjana, N. E., Koralov, S. B. and Smibert, P. Multiplexed detection of proteins, transcriptomes, clonotypes and CRISPR perturbations in single cells. Nature Methods 16, 409-412, doi:10.1038/s41592-019-0392-0 (2019).
19. Buus, T. B., Herrera, A., Ivanova, E., Mimitou, E., Cheng, A., Herati, R. S., Papagiannakopoulos, T., Smibert, P., Odum, N. and Koralov, S. B. Improving oligo-conjugated antibody signal in multimodal single-cell analysis. eLife 10, doi:10.7554/eLife.61973 (2021).
20. Klemm, S. L., Shipony, Z. and Greenleaf, W. J. Chromatin accessibility and the regulatory epigenome. Nature Reviews Genetics 20, 207-220, doi:10.1038/s41576-018-0089-8 (2019).
21. Satpathy, A. T., Granja, J. M., Yost, K. E. et al. Massively parallel single-cell chromatin landscapes of human immune cell development and intratumoral T cell exhaustion. Nature Biotechnology 37, 925-936, doi:10.1038/s41587-019-0206-z (2019).
22. Iyer, V. R. Nucleosome positioning: bringing order to the eukaryotic genome. Trends Cell Biol 22, 250-256, doi:10.1016/j.tcb.2012.02.004 (2012).
23. Lara-Astiaso, D., Weiner, A., Lorenzo-Vivas, E., Zaretsky, I., Jaitin, D. A., David, E., Keren-Shaul, H., Mildner, A., Winter, D., Jung, S., Friedman, N. and Amit, I. Immunogenetics. Chromatin state dynamics during blood formation. Science 345, 943-949, doi:10.1126/science.1256271 (2014).
24. Saeed, S., Quintin, J., Kerstens, H. H. et al. Epigenetic programming of monocyte-to-macrophage differentiation and trained innate immunity. Science 345, 1251086, doi:10.1126/science.1251086 (2014).
25. Luger, K., Mader, A. W., Richmond, R. K., Sargent, D. F. and Richmond, T. J. Crystal structure of the nucleosome core particle at 2.8 A resolution. Nature 389, 251-260, doi:10.1038/38444 (1997).
26. Jiang, C.and Pugh, B. F. Nucleosome positioning and gene regulation: advances through genomics. Nature Reviews Genetics 10, 161-172, doi:10.1038/nrg2522 (2009).
27. Buenrostro, J. D., Giresi, P. G., Zaba, L. C., Chang, H. Y. and Greenleaf, W. J. Transposition of native chromatin for fast and sensitive epigenomic profiling of open chromatin, DNA-binding proteins and nucleosome position. Nature Methods 10, 1213-1218, doi:10.1038/nmeth.2688 (2013).
28. Lareau, C. A., Duarte, F. M., Chew, J. G., Kartha, V. K., Burkett, Z. D., Kohlway, A. S., Pokholok, D., Aryee, M. J., Steemers, F. J., Lebofsky, R. and Buenrostro, J. D. Droplet-based combinatorial indexing for massive-scale single-cell chromatin accessibility. Nature Biotechnology 37, 916-924, doi:10.1038/s41587-019-0147-6 (2019).
29. De Simone, M., Rossetti, G. and Pagani, M. Single Cell T Cell Receptor Sequencing: Techniques and Future Challenges. Front Immunol 9, 1638, doi:10.3389/fimmu.2018.01638 (2018).
30. Stoeckius, M., Zheng, S., Houck-Loomis, B., Hao, S., Yeung, B. Z., Mauck, W. M., Smibert, P. and Satija, R. Cell Hashing with barcoded antibodies enables multiplexing and doublet detection for single cell genomics. Genome Biology 19, 224, doi:10.1186/s13059-018-1603-1 (2018).
31. Herrera, A., Cheng, A., Mimitou, E. P., Seffens, A., George, D., Bar-Natan, M., Heguy, A., Ruggles, K. V., Scher, J. U., Hymes, K., Latkowski, J. A., Odum, N., Kadin, M. E., Ouyang, Z., Geskin, L. J., Smibert, P., Buus, T. B. and Koralov, S. B. Multimodal single-cell analysis of cutaneous T-cell lymphoma reveals distinct subclonal tissue-dependent signatures. Blood 138, 1456-1464, doi:10.1182/blood.2020009346 (2021).
32. Zavitsanou, A.-M., Hao, Y., Wu, W. L. et al. KEAP1 mutation in lung adenocarcinoma promotes immune evasion and immunotherapy resistance. bioRxiv, 2021.2009.2024.461709, doi:10.1101/2021.09.24.461709 (2021).
33. Curcio, M. J.and Derbyshire, K. M. The outs and ins of transposition: from mu to kangaroo. Nat Rev Mol Cell Biol 4, 865-877, doi:10.1038/nrm1241 (2003).
34. Finnegan, D. J. Eukaryotic transposable elements and genome evolution. Trends Genet 5, 103-107, doi:10.1016/0168-9525(89)90039-5 (1989).
35. Kleckner, N. Transposable elements in prokaryotes. Annu Rev Genet 15, 341-404, doi:10.1146/annurev.ge.15.120181.002013 (1981).
36. Adey, A. C. Tagmentation-based single-cell genomics. Genome Res 31, 1693-1705, doi:10.1101/gr.275223.121 (2021).
37. Yan, F., Powell, D. R., Curtis, D. J. and Wong, N. C. From reads to insight: a hitchhiker's guide to ATAC-seq data analysis. Genome Biology 21, 22, doi:10.1186/s13059-020-1929-3 (2020).
38. Gradman, R. J.and Reznikoff, W. S. Tn5 synaptic complex formation: role of transposase residue W450. J Bacteriol 190, 1484-1487, doi:10.1128/jb.01488-07 (2008).
39. Yang, S., Corbett, S. E., Koga, Y., Wang, Z., Johnson, W. E., Yajima, M. and Campbell, J. D. Decontamination of ambient RNA in single-cell RNA-seq with DecontX. Genome Biology 21, 57, doi:10.1186/s13059-020-1950-6 (2020).
40. Merrill, C. B., Pabon, M. A., Montgomery, A. B., Rodan, A. R. and Rothenfluh, A. Optimized assay for transposase-accessible chromatin by sequencing (ATAC-seq) library preparation from adult Drosophila melanogaster neurons. Scientific Reports 12, 6043, doi:10.1038/s41598-022-09869-4 (2022).
41. DeAngelis, M. M., Wang, D. G. and Hawkins, T. L. Solid-phase reversible immobilization for the isolation of PCR products. Nucleic Acids Res 23, 4742-4743, doi:10.1093/nar/23.22.4742 (1995).
42. Bray, N. L., Pimentel, H., Melsted, P. and Pachter, L. Near-optimal probabilistic RNA-seq quantification. Nature Biotechnology 34, 525-527, doi:10.1038/nbt.3519 (2016).
43. Melsted, P., Booeshaghi, A. S., Liu, L., Gao, F., Lu, L., Min, K. H., da Veiga Beltrame, E., Hjarleifsson, K. E., Gehring, J. and Pachter, L. Modular, efficient and constant-memory single-cell RNA-seq preprocessing. Nature Biotechnology 39, 813-818, doi:10.1038/s41587-021-00870-2 (2021).
44. Stuart, T., Butler, A., Hoffman, P., Hafemeister, C., Papalexi, E., Mauck, W. M., 3rd, Hao, Y., Stoeckius, M., Smibert, P. and Satija, R. Comprehensive Integration of Single-Cell Data. Cell 177, 1888-1902.e1821, doi:10.1016/j.cell.2019.05.031 (2019).
45. Giansanti, V., Tang, M. and Cittaro, D. Fast analysis of scATAC-seq data using a predefined set of genomic regions. F1000Res 9, 199, doi:10.12688/f1000research.22731.2 (2020).
46. Hao, Y., Hao, S., Andersen-Nissen, E. et al. Integrated analysis of multimodal single-cell data. Cell 184, 3573-3587.e3529, doi:https://doi.org/10.1016/j.cell.2021.04.048 (2021).
47. Ivanova, E. N., Devlin, J. C., Buus, T. B., Koide, A., Shwetar, J., Cornelius, A., Samanovic, M. I., Herrera, A., Mimitou, E. P., Zhang, C., Desvignes, L., Odum, N., Smibert, P., Ulrich, R. J., Mulligan, M. J., Koide, S., Ruggles, K. V., Herati, R. S. and Koralov, S. B. SARS-CoV-2 mRNA vaccine elicits a potent adaptive immune response in the absence of IFN-mediated inflammation observed in COVID-19. medRxiv, 2021.2004.2020.21255677, doi:10.1101/2021.04.20.21255677 (2021).
48. Gerard G. F., a. D. A. J. M. in Methods in Molecular Biology Vol. Vol.16 (ed M. M. Burell) 73-93 (Humana Press Inc., 1993).
49. Xu, Z., Heidrich-O'Hare, E., Chen, W. and Duerr, R. H. Comprehensive benchmarking of CITE-seq versus DOGMA-seq single cell multimodal omics. Genome Biology 23, 135, doi:10.1186/s13059-022-02698-8 (2022).
50. Institute, B. Picard toolkit, <http://broadinstitute.github.io/picard/>
51. Liu, B., Li, C., Li, Z., Wang, D., Ren, X. and Zhang, Z. An entropy-based metric for assessing the purity of single cell populations. Nature Communications 11, 3155, doi:10.1038/s41467-020-16904-3 (2020).
52. Bartosovic, M.and Castelo-Branco, G. Multimodal chromatin profiling using nanobody-based single-cell CUT&amp;amp;Tag. bioRxiv, 2022.2003.2008.483459, doi:10.1101/2022.03.08.483459 (2022).
   All patents, applications, publications, test methods, literature, and other materials cited herein are hereby incorporated by reference in their entirety as if physically present in this specification.
   While several possible embodiments are disclosed above, embodiments of the present invention are not so limited. These exemplary embodiments are not intended to be exhaustive or to unnecessarily limit the scope of the invention, but instead were chosen and described in order to explain the principles of the present invention so that others skilled in the art may practice the invention. Indeed, various modifications of the invention in addition to those described herein will become apparent to those skilled in the art from the foregoing description. Such modifications are intended to fall within the scope of the appended claims.

1-14. (canceled)

15. A method for simultaneously measuring the 5′ end of RNA transcripts and chromatin accessibility at the single-cell level in a biological sample comprising one or more cells, the method comprising:
a) permeabilizing the cells in the biological sample;
b) incubating the permeabilized cells with a plurality of transposome assemblies each comprising a transposase, a first adaptor, and a second adaptor comprising a 3′ single-stranded overhang on the non-transfer strand, wherein the transposase makes double-strand breaks in genomic DNA and attaches the first and second adaptors at the ends of the genomic DNA fragments that are produced,
thereby producing tagmented genomic DNA fragments flanked on both sides by a transposase recognition site and a sequence complementary to a capture sequence of a third oligonucleotide, the complementary sequence located at a single stranded 3′ end of the non-transfer strand of tagmented genomic DNA, or flanked on one side by a transposase recognition site and a sequence complementary to a capture sequence of a third oligonucleotide, the complementary sequence located at a single stranded 3′ end of the non-transfer strand of tagmented genomic DNA, and by a transposase recognition site and a unique sequence on the other side;
c) incubating the permeabilized cells with a carrier comprising the third oligonucleotide comprising one or more of a barcode sequence, a cell barcode sequence, and a unique molecular identifier (UMI);
d) separating individual cells, each separated cell comprising the carrier comprising the third oligonucleotide, mRNA molecules, and tagmented genomic DNA fragments;
e) generating cDNA from the mRNA molecules, the cDNA being captured by the third oligonucleotide attached to the carrier;
f) purifying the cDNA and tagmented genomic DNA fragments;
g) performing gap-filling on the tagmented genomic DNA fragments;
h) ligating to seal nicks in the gap-filled tagmented genomic DNA fragments;
i) amplifying the cDNA and the ligated gap-filled tagmented genomic DNA fragments with primers and separating the amplified cDNA from the amplified genomic DNA fragments;
j) preparing a cDNA library from the amplified cDNA; and
k) preparing an assay for transposase-accessible chromatin (ATAC) library from the amplified genomic DNA fragments.

16. The method of claim 15, wherein the transposase is a Tn5 transposase or a hyperactive Tn5 transposase or a functional mutant or derivative of any said transposase, or a MuA transposase or a functional mutant or derivative thereof, and wherein the transposase recognition site is a Tn5 recognition site or a MuA recognition site comprising a R1 sequence and a R2 sequence.

17. The method of claim 16, wherein the transposase is a Tn5 transposase or a hyperactive Tn5 transposase or a functional mutant or derivative thereof, and wherein the transposase recognition site is the Tn5 recognition site.

18. The method of claim 15, wherein step (a) and step (b) are performed sequentially or simultaneously.

19. (canceled)

20. The method of claim 15, wherein step (g) and step (h) are performed sequentially or simultaneously.

21. (canceled)

22. The method of claim 15, wherein the method comprises generating the transposome assembly comprising the transposase, the first adaptor, and the second adaptor prior to step (b), and wherein generating the transposome assembly comprises:
providing a first oligonucleotide comprising a free 5′ phosphate at a 5′ end of the first oligonucleotide and the transposase recognition site;
providing a second oligonucleotide comprising a free 5′ phosphate at a 5′ end of the second oligonucleotide, the transposase recognition site, and the sequence complementary to the capture sequence of the third oligonucleotide at the 3′ end of the second oligonucleotide;
providing a fourth oligonucleotide comprising a free 5′ phosphate at a 5′ end of the fourth oligonucleotide, the transposase recognition site, and a unique sequence at the 3′ end of the fourth oligonucleotide;
annealing the first oligonucleotide and the second oligonucleotide to one another to form the first adaptor that contains a double-stranded transposase recognition site and a single-stranded 3′ overhang on a non-transfer strand, the single-stranded 3′ overhang comprising the sequence complementary to the capture sequence of the third oligonucleotide;
annealing the first oligonucleotide and fourth oligonucleotide to one another to form the second adaptor that contains a double-stranded transposase recognition site and a single-stranded 3′ overhang on a non-transfer strand, the single-stranded 3′ overhang comprising the unique sequence; and
incubating the first adaptor and the second adaptor with the transposase to form the transposome assembly.

23. (canceled)

24. The method of claim 22, wherein the second oligonucleotide comprises a modification at the 3′ end to prevent degradation of the oligonucleotide, optionally wherein the modification is one or more phosphorothioate bonds.

25. The method of claim 15, wherein the carrier is a bead or a solid surface.

26. The method of claim 15, wherein the method further comprises prior to step (a), labeling the cells in the biological sample with oligonucleotide-labeled lipids, oligonucleotide-tagged hashing antibodies, or oligonucleotide-tagged antibodies against specific surface markers.

27. The method of claim 26, wherein the cells are labeled with the oligonucleotide-labeled lipids the oligonucleotide-tagged hashing antibodies, or the oligonucleotide-tagged antibodies or against specific surface markers, the method comprising:
generating antibody-derived tag (ADT) fragments from the oligonucleotide-tagged hashing antibodies, or the oligonucleotide-tagged antibodies, the ADT fragments being captured by the third oligonucleotide attached to the carrier;
purifying the ADT fragments;
separating the ADT fragments from the amplified genomic DNA fragments and/or cDNA;
amplifying the ADT fragments; and
preparing an ADT library from the amplified ADT fragments.

28. The method of claim 15, wherein:
step (a) further comprises incubation with a RNase inhibitor;
step (b) further comprises inactivating the transposase; and/or
step (c) further comprises counting the number of cells in the biological sample, resuspending the cells at a concentration of 150-1500 cells/ml, and processing the resuspended cells for bead-based single-cell RNA-sequencing.

29. (canceled)

30. (canceled)

31. The method of claim 15, wherein generating cDNA in step (e) comprises:
reverse transcribing the mRNA with a reverse transcriptase enzyme to generate the cDNA fragments; and
inactivating the reverse transcriptase enzyme.

32. The method of claim 31, wherein the transposase is inactivated by heating to 65° C., incubating with 0.04% SDS at room temperature, or by the addition of EDTA.

33. The method of claim 15, wherein:
step (f) comprises forming a plurality of droplets, each droplet comprising an individual cell,
the gap-filling in step (g) is performed by a non-hot start non-gap filling DNA polymerase and/or
wherein the ligation in step (h) is performed by a T4 DNA ligase or an E. coli DNA ligase.

34. (canceled)

35. (canceled)

36. The method of claim 15, wherein the cDNA is used to amplify a V(D)J region of transcripts using specific primers to generate a library of V(D)J fragments.

37. The method of claim 36, wherein the cDNA library, ADT library, V(D)J library, and ATAC library are pooled after they are generated.

38. (canceled)

39. The method of claim 37, wherein the biological sample is characterized based upon the sequencing readout.

40. The method of claim 39, wherein the biological sample comprises a single cell, and the single readout comprises a single cell readout.

41. The method of claim 36, further comprising associating a cDNA fragment, an ADT fragment, a V(D)J fragment, and/or genomic DNA fragment with a single cell in the biological sample based on the cell barcode sequence of the third oligonucleotide.

42. The method of claim 15, wherein after a sequencing analysis, the biological sample is assigned to a sample of origin based on a barcode sequence of the carrier and/or of an oligonucleotide-tagged hashing antibody, an oligonucleotide-tagged hashing antibody or an oligonucleotide-tagged lipid in the ADT library, and wherein the sequencing analysis is analyzed to generate a representation of accessible chromatin, a transcriptomic profile, an immune receptor repertoire, and/or a surface epitope repertoire of a single cell in the biological sample.

43-191. (canceled)


SEQUENCE LISTING (6 sequences)

SEQ ID NO: 1 - DNA, 19 bp, synthetic construct
  source 1..19: mol_type = other DNA
  modified_base 1: mod_base = OTHER; note = 5' Phosphate modified nucleotide
ctgtctcttatacacatct

SEQ ID NO: 2 - DNA, 32 bp, synthetic construct
  source 1..32: mol_type = other DNA
  modified_base 1: mod_base = OTHER; note = 5' Phosphate modified nucleotide
  modified_base 30: mod_base = OTHER; note = Nucleotide with phosphorothioate linkage
  modified_base 31: mod_base = OTHER; note = Nucleotide with phosphorothioate linkage
ctgtctcttatacacatctcccatataagaaa

SEQ ID NO: 3 - DNA, 28 bp, synthetic construct
  source 1..28: mol_type = other DNA
  modified_base 1: mod_base = OTHER; note = 5' Phosphate modified nucleotide
  modified_base 28: mod_base = OTHER; note = Desthiobiotin-triethylene glycol modified nucleotide
ctgtctcttatacacatctcccatataa

SEQ ID NO: 4 - DNA, 19 bp, synthetic construct
  source 1..19: mol_type = other DNA
  modified_base 1: mod_base = OTHER; note = 5' Phosphate modified nucleotide
agatgtgtataagagacag

SEQ ID NO: 5 - protein, 13 aa, Homo sapiens
CAGSLYNFNKFYF

SEQ ID NO: 6 - protein, 15 aa, Homo sapiens
CASSRQGGSGNTIYF